(19) United States
(12) Patent Application Publication
(10) Pub. No.: US 2009/0143655 A1
(43) Pub. Date: Jun. 4, 2009

(54) Apparatus, System and Method for Determining Cardio-Respiratory State

(76) Inventor: Haim Shani, Shoham (IL)

Correspondence Address: THE NATH LAW GROUP, 112 South West Street, Alexandria, VA, 22314, US

(21) Appl. No.: 11/992,921

(22) PCT Filed: Jan. 30, 2007
(86) PCT No.: PCT/IL2007/000114

§ 371 Date: Apr. 1, 2008

Related U.S. Application Data
(60) Provisional application No. 60/762,892, filed on Jan. 30, 2006.

(51) Int. Cl. A61B 5/145; A61B 5/02; A61B 6/00; A61B 8/00

(52) U.S. Cl. 600/323; 600/484; 600/473; 600/454

(57) ABSTRACT

An apparatus, system and method provide data indicative of cardio-respiratory state of a patient. Two or more cardio-respiratory parameters of the patient are measured, and optionally monitored over time, the two or more cardio-respiratory parameters being different one from the other and being measured at a same anatomical part of said patient.

### FIELD OF THE INVENTION

This invention relates generally to the diagnosis of cardio-respiratory status and shock, and to methods and devices for carrying out the diagnosis. More particularly, the invention relates to methods, systems and apparatuses for the non-invasive determination of cardio-respiratory status.

### BACKGROUND OF THE INVENTION

Diagnosis of cardio-respiratory state of a patient is an important tool in the health care of some patients. Particular distortions of the cardio-respiratory state can indicate the early stages of potentially life threatening conditions, for example dehydration or shock, as well as deterioration of life signs of the patient.

Herein, the term “cardio-respiratory parameter” relates to any parameter that is related to the cardio-respiratory system of the body, including for example blood perfusion, peripheral blood perfusion (for example capillary refill time), respiratory rate, blood pressure, pulse rate, and so on.

Herein “blood perfusion” refers to blood flow, particularly of red blood cells, through the organs and tissues of the body. Body organs and tissues have to be supplied with oxygen and different substances in order to provide the metabolism of cellular tissue. This supply is provided through the vascular system by the flow of blood. This flow, passing through the blood vessels and capillaries of tissues of the peripheral parts of the body, is referred to as peripheral blood perfusion.

Common changes in physiological state of body, such as trauma or dehydration for example, can cause the reduction of the blood flow in the peripheral regions of the body, such as for example the fingers or other extremities, and subsequently this effect is reflected in the decreasing of peripheral blood perfusion, supplying less oxygen and substances to the tissues.

As cells are starved by oxygen and substances, the cells can no longer sustain efficient aerobic oxygen production. Aerobic metabolism generates thirty six adenosine triphosphate (ATP) molecules per glucose molecule. As oxygen delivery is impaired, the cell must switch to the much less efficient anaerobic metabolic pathway, which generates only two ATP molecules per molecule of glucose, with resulting production and accumulation of lactic acid. Eventually, cellular metabolism is no longer able to generate enough energy to power the components of cellular homeostasis, leading to the disruption of cell membrane ionic pumps, accumulation of intracellular sodium with an efflux of potassium, and accumulation of cytosolic calcium. The cell swells, the cell membrane breaks down, and cell death ensues. Widespread cellular death results in multiple system organ failure and, if irreversible, in patient death.

Other factors can also cause a change in peripheral blood perfusion—such as drugs, vascular diseases, transplantations and surgery, intravascular infusion, etc. These factors may be local (vascular diseases, transplantations and local surgery, etc) or remote (shock, drugs, diabetic disorders, etc) in character.

Monitoring and diagnosing of peripheral blood perfusion is a useful indicator of the global haemodynamic physiological state, such as shock, or of local or systemic cardiorespiratory pathology.

Expressed in its simplest terms, shock is the consequence of an inadequate delivery of blood or liquids to a major organ of the human body. Unless shock is promptly treated, this deprivation of blood may give rise to a disturbance in the metabolism of the organ with a resultant damage thereto. Because of the serious consequences of shock or dehydration, its detection and treatment is regarded medically as an emergency procedure in which time is of the essence.

Cellular damage to an organ may be reversed by prompt treatment of shock. But it is otherwise irreversible and may lead to the death of the patient. Recovery from shock therefore depends on the promptness of treatment. However, before a patient can be treated for shock he must first be diagnosed to determine whether the patient is actually experiencing early shock or shock or any other cardiorespiratory disturbance.

The treatment to be administered to a patient in shock depends on the nature of his condition. For example, for some shock conditions the appropriate treatment includes fluid resuscitation and drugs such as dopamine which acts to increase arterial perfusion pressure. Treatment for a shock condition must be administered with extreme care while the patient is being monitored.

Medical authorities classify shock syndrome in the following five categories:

(1) Hypovolemic shock

(2) Septic shock

(3) Cardiogenic shock

(4) Obstruction to cardiac filling shock

(5) Neurogenic shock

Hypovolemic shock, the most common type of shock, is caused by a massive loss of blood, plasma or fluid from the body of a patient, or the loss of fluid from an intravascular compartment. Such losses may be due to dehydration, vomiting, diarrhea, burns, or because of the use of diuretics. A loss of blood and plasma is experienced in hemorrhagic shock such as in cases of blunt and penetrating trauma injuries, gastrointestinal bleeding, or Gynecologic/Obstetric bleeding. Many cases of bleeding are occult (e.g. slow internal bleeding), and therefore can not be diagnosed early.

Septic shock is caused by bacterial infection in which an endotoxin is released into the blood stream. The sequestration and pooling of blood in various vascular compartments reduces the availability of blood for the perfusion of other vital organs.

Cardiogenic shock is usually attributed to a massive myocardial infarction caused by extensive damage to the myocardium. This may be the result of arrhythmia in a patient suffering from heart disease. In this category of shock syndrome, the heart fails to pump properly, with a consequent reduction in arterial blood.

Obstruction to cardiac filling shock takes place when this filling activity is lessened or arrested by a massive pulmonary embolism, or by space-occupying lesions. Neurogenic shock results from a severe spinal cord injury, or from a massive intake of a depressant drug, causing a loss of vasometric tone.

The five categories of shock syndrome each represent other causes of cardio-pulmonary distress, or a shock-related condition. The term “shock-related condition”, as used hereinafter, is intended to embrace all five categories.

Known non-invasive methods to diagnose shock do not evaluate perfusion. These methods rely on the following cardiovascular parameters:

Blood pressure. The measurement of blood pressure clearly identifies shock only in its late stages when blood pressure drops significantly (uncompensated shock).

Heart rate or Pulse rate. The specificity of this measurement is low because heart rate is also increased by other common physiological conditions, such as anxiety and pain.

Capillary Refill Time (CRT). When applying pressure onto a specific skin area, the capillaries below the depressed area collapse and blood is blanched therefrom, thereby causing the skin color in the depressed skin area to whiten. Upon abrupt release of this pressure, blood flows back into the capillaries and the original skin color is recovered. CRT is defined as the time it takes for the original pink skin color to return after it had been blanched. In clinical practice, prolongation of the CRT for more than 2 second is considered to reflect poor skin perfusion, usually associated with systemic hypoperfusion or shock. This well-known bed-side test, although subjective and inaccurate, is an important vital sign of a shock state. If an appropriate treatment has not been given early enough, the shock condition will continue to deteriorate, the arteriolar and capillary vasoconstriction will increase even further, as reflected by prolongation of the CRT, blood pressure will fall, and the patient may die. However, an appropriate prompt treatment at the early stage of shock will decrease vasoconstriction and shorten the CRT. In U.S. Pat. No. 6,685,635, assigned to the present assignee and the contents of which are incorporated herein, describes an instrument for determining CRT, comprising a color sensor trained on the skin area and responsive to light reflected therefrom to produce a first signal at the point in time the skin color turns from pink to white and to later produce a second signal at the point in time at which the skin color has turned from white to pink. The time elapsing between the first and second signals is measured to provide a CRT index indicative of the patient's condition.

There are also relatively complex, expensive and difficult to interpret clinical techniques for providing a measure of blood perfusion, laser Doppler devices for example. Time is of the essence in the diagnosis and treatment of shock, yet known types of skin capillary flow instrumentation are incapable of facilitating rapid diagnosis and treatment of shock. It is vital that skin capillary flow instruments have a high order of accuracy so that their readings indicate the severity of the shock or shock-related condition.

Studies published in the medical literature over the last two years demonstrate that skin temperature independently influences the skin capillary flow. One major limitation of prior skin capillary flow measurement devices is that they do not take into account skin temperature, and therefore do not correlate the measurement to skin temperature.

“Perfusion” refers to blood flow through the organs and tissues of the body, and thus a perfusion based or dependent parameter is a parameter that varies in a dependent manner with respect to the flow of blood through a tissue organ.

Perfusion based or perfusion dependent parameters (PU) are sometimes used for cardiovascular diagnostics. Such parameters include, for example, perfusion index (PI), concentration of moving blood cells (CMBC), perfusion impedance, and so on, and may be determined using known methods such as photoplethysmography, impedance plethysmography, vascular ultrasonography, Doppler ultrasonography, Doppler optical flowmetry and so on. In Doppler optical flowmetry, for example, microvascular blood perfusion, i.e. red blood cell flux through a microvasculature is defined as the product of the number of blood cells moving in a tissue sampling volume, and the mean velocity of these cells in the sampling volume. Such a parameter is typically measured in relative units known as blood perfusion units—designated BPU or more simply as PU. The absolute magnitude of this parameter varies from patient to patient, and from measurement region to measurement region for the same patient, essentially because the sample volume is undefined and thus varies with patient and location on the patient.

### SUMMARY OF THE INVENTION

In accordance with the present invention, there is provided an apparatus, system and method, to determine the cardiorespiratory state of a patient, in particular to measure and monitor the severity of this physiologic condition, for example at specific points in time or as an on-going monitoring process with respect to a patient. The present invention thus facilitates diagnosis of such a cardiorespiratory state of a patient, and in some embodiments helps to detect shock-related conditions, in a non-invasive manner.

The present invention thus relates to an apparatus for providing data indicative of cardio-respiratory state of a patient, the apparatus comprising at least two cardio-respiratory sensors in the form of sensor modules for providing at least two cardio-respiratory parameters, including:—

first sensor module for measuring a first cardio-respiratory parameter of said patient;

second sensor module for measuring a second cardio-respiratory parameter of said patient, different from said first cardio-respiratory parameter;

wherein said apparatus is adapted for measuring said first cardio-respiratory parameter and said second cardio-respiratory parameter at a same anatomical part of said patient.

In some embodiments, the apparatus is particularly adapted for the diagnosis of any one of shock, early shock and dehydration.

In some applications, the same anatomical part may comprise a skin portion and/or may comprise an extremity, optionally including any one of: nose, ear, finger, hand, arm, toe, foot, leg of a patient, for example.

In some embodiments, the apparatus optionally further comprises a third cardio-respiratory sensor module for measuring at said same anatomical part at least one third cardio-respiratory parameter of said patient different from said first or second cardio-respiratory parameters.

In other embodiments, the apparatus may optionally further comprises a fourth cardio-respiratory sensor module for measuring at said same anatomical part at least one fourth cardio-respiratory parameter of said patient different from said first, second or third cardio-respiratory parameters.

Each said cardio-respiratory sensor may be configured for monitoring a different one of any of the following cardio-respiratory parameters: capillary refill time (CRT); a peripheral perfusion parameter other than CRT; blood oxygenation level; blood pressure; pulse rate; systemic vascular resistance. At least two said cardio-respiratory sensors may be configured for measuring corresponding cardio-respiratory parameters with respect to a common vascular bed on said same anatomical part, and/or, at least two said cardio-respiratory sensors are configured for measuring corresponding cardio-respiratory parameters substantially simultaneously, and/or at least two said cardio-respiratory sensors are configured for monitoring corresponding cardio-respiratory parameters over a predetermined period of time.

When providing such measurements from the same vascular bed, this may permit the doctor or other caregiver to infer about both the arterial and capillary tones simultaneously.

In some embodiments, one said cardio-respiratory sensors comprises a CRT sensor module configured for monitoring a capillary refill time (CRT), said CRT sensor module comprising:

means for illuminating a skin area comprised in said same anatomical part to be gauged for wavelength with a light from a light source;

means for filtering out background noises and light to obtain a base-line measurement; and

means for comparing the wavelength of light received from the skin area with the base-line measurement, thereby determining the filling time of blood vessels in said area.

In some embodiments, one said cardio-respiratory sensors comprises a CRT sensor module configured for monitoring a capillary refill time (CRT), said CRT sensor comprising:

a light source for illuminating a skin area of the patient's skin overlying blood vessels with light at a first wavelength, said skin area having an original color (i.e., wavelength, in the visible or invisible spectrum), a light sensor for intercepting light at a second wavelength obtained from said skin area or associated with a depth within said skin area and generating a first signal having a magnitude which corresponds to the second wavelength, said second wavelength representing a level of reflection from blood vessels subjacent said skin area;

a filter for filtering said first electrical signal and for rejecting unwanted electrical signals originating in interfering light, and for producing a second signal, whose amplitude is proportional to the amplitude of said filtered first signal;

means for storing the amplitude value of said second signal which corresponds to said original color;

a transducer for applying pressure on said skin area, and for obtaining an amplitude of the second signal which corresponds to maximum whitening of said skin area.

This embodiment may optionally further comprise a processor for processing data collected by said transducer and for measuring the filling time of blood vessels after releasing said pressure.

Regarding the CRT sensor module, the light from said light source may be substantially modulated or substantially non-modulated. Optionally, the apparatus may further include means for sampling the amplitude value of the second electrical signal at a predetermined rate during the measurement and for storing said sampled values. The second measuring means may be adapted for basing said first signal and said second signal on a portion of said area of skin close to but not including the part of the skin that is directly pressured by said transducer.

Optionally, said measuring the filling time of blood vessels after releasing said pressure is provided by analysing a rate of change of light intensity of said second wavelength with respect to elapsed time after releasing said pressure. Further optionally, a suitable mechanism for automatically applying and releasing said pressure, for example via a suitable mechanical pneumatic, hydraulic, magnetic or electrical actuation arrangement.

When measuring CRT it is essential that pressure be applied only to capillary vessels while maintaining normal blood flow. In an embodiment of a system in accordance with the invention, a programmable mechanical unit applies an accurate measurable amount of pressure to the skin.

Optionally, the apparatus comprising said CRT sensor module further comprises a first temperature sensor for sensing skin temperature of a second skin area close to said first mentioned skin area, wherein said second skin area is substantially unaffected by heat effects generated by said apparatus. The apparatus may further comprise a second temperature sensor for sensing skin temperature of said first mentioned area, wherein said first mentioned skin area is substantially unaffected by heat effects generated by said apparatus.

The apparatus optionally further comprises correction means for correcting said amplitude of said second signal to compensate for effects that may be caused by skin movement after said releasing of pressure. The correction means may include, for example, a suitable algorithm embodied in said processor. The transducer may comprise means for determining parameters including skin resistance to pressure as a function of depression of the skin responsive to the action of said transducer, and wherein said parameters are provided as inputs to said algorithm. Optionally, the CRT sensor module may be adapted for maintaining a substantially constant skin-to-light sensor displacement during operation thereof.

Some embodiments of the system of the invention which incorporate a CRT sensor module include a color sensor trained on the skin area and responsive to light reflected therefrom to produce a first signal at the point in time the depressed skin color is blanched from pink to white and pressure is released when blanching at minimal pressure is attained, to later produce a second signal at the point in time at which the skin color regains its natural pink color. Herein, “color sensor” refers to any light sensor capable of sensing intensities of light within any desired range of wavelengths, for example the full range of visible light, or any other range of wavelengths, either within the visible range, beyond the same or overlapping both, among others. When the post-blanching skin color corresponds to a pre-test natural color, the CRT can be detected by recording the time which has elapsed from the maximal blanching point to this final point. In other words, the time elapsing between the first signal (starting point of minimal blanching pressure release) and the second signal (final point where post-blanching color equals pre-test color) is measured to provide a CRT index indicative of the patient's condition at the time the test was conducted.

For each pre-determined time interval, this measurement is repeated and a new CRT is recorded.

The device can continue measuring CRT at any desired interval, for example every 30 seconds to 1-10 minutes (this depends on clinical demands), and a change of CRT over time will be recorded and monitored.

Concurrently, other cardiovascular parameters, for example blood oxygenation or parameters derived from blood pressure or pulse blood pressure measurements may also be monitored at the same site.

Optionally, the CRT data may be corrected for distance effects introduced by the displacement of the skin during spring-back from the depressed position during CRT testing. Alternatively, the apparatus may be configured to minimize such distance effects. Optionally, the CRT data may be adjusted to take account of the temperature of the patient. Further, heating effects due to the apparatus itself may also be compensated for.

Optionally, potentially false color readings originating from capillary damage due to repeated testing of a skin area may be avoided by sensing the color changes in an area close to but not including the area of skin that is being directly pressured by the apparatus of the invention.

FIG. 11 is a graphical representation of CRT measurement results. At the first stage, no pressure is applied on the skin, and therefore the system of the invention can carry out calibration of the initial skin color of the patient. The value of the calibration is stored for use at the end of the measurement. The calibration process is essential in that the normal color of the skin depends on the individual and differs from patient to patient.

At the second stage of operation, pressure is applied to the skin at a magnitude and for a duration sufficient to obtain maximum whitening of the skin color in the depressed area. The processor can be programmed to provide a visual and/or audio warning signal (such as a beep, for example) to the user when the pressure is insufficient or shorter in duration than required. Obtaining maximum whitening of all the depressed area is indicative of sufficient whitening pressure.

Stronger pressures of longer duration do not affect the skin color beyond maximum whitening. After obtaining maximum whitening, a signal indicative thereof is provided to the user to quickly release the pressure. Measurement of the CRT is started at that instant (to) at which the skin coloring proceeds to change from its maximum whitening color to regain its original pinkish color. Normally, the rate of filling is higher at the beginning of the filling process and lower as time lapses.

The system uses the stored calibration value to determine the moment tf at which the normal pink skin color is regained, at which point the measurement ceases. The recovery time can be determined by the desired degree of measurement accuracy. For example, point tf can be defined as the instant at which the value of the digital word that corresponds to the current skin color reaches a value that is 90% of the value of the digital word that corresponds to the original skin color of the patient being diagnosed. In the graph of FIG. 11, the CRT reading is given by t_{f}-tₒ.

The accuracy of the CRT measurement can also be determined by the rate of change in the skin coloring in the time interval that is close to the conclusion of the measurement. The last segment of the graph lies between the points of time t₁ and t_{f}. The rate of change in this time interval is nearly constant and is nearly insensitive to the magnitude and duration of the applied pressure. Hence, the CRT can be extrapolated with relatively high accuracy from the time interval t_{f}-t₁. Under normal conditions CRT should be below one second. A CRT value above two seconds can be regarded as representing a pre-shock state. Longer CRT values can be considered to be indicative of more severe shock states.

The accuracy of the measurement can also be determined by the rate of change in the skin coloring, in the time interval that is close to the completion of the measurement. The last segment of the graph appears between the time points t₁ and t_{f}. The rate of change in this time interval is nearly constant, and is almost insensitive to the magnitude and duration of the applied pressure. Hence the CRT can be extrapolated with relative accuracy from the time interval t_{f}-t₁.

The CRT under normal shock-free conditions should be below 1 second. When a CRT value rising above 2 seconds is diagnosed. This is indicative of a pre-shock state. Longer CRT values indicate a more severe shock condition.

FIG. 12 is a graphical representation of the CRT as a function of shock-state for obtaining inferences related to the trend of the patient's physiological condition in response to medical treatment. In the initial time interval between time-points t₂ and t₃, the CRT value is then below 2 seconds, hence the patient is in a normal, shock-free condition. An early and mild shock condition starts at time-point t₃ where the CRT value exceeds 2 seconds. As time lapses with no proper treatment of the shock condition, the shock becomes more severe until time-point t₄ is reached. This point indicates the entry of the patient into a moderate shock condition (CRT value higher than 3 seconds). The next stage is indicated by the time-point t₅. This indicates the entry of the patient into a late (severe) shock condition (CRT value higher than 4 seconds). From point t₅ and beyond, the CRT rises rapidly.

Referring to FIG. 13, example results using the system of the present invention are illustrated, wherein the squares represent CRT data, and the curve represents PU data. A CRT threshold can be defined, say 1.3 seconds, illustrated as a broken line in FIG. 13, wherein lower values are considered to be within norm, and lower values, out of norm. In the illustrated example of FIG. 13, there are a first and third regions, A1, A3 which are out of norm, and an intermediate region A2 which is within norm.

Analysis of skin temperature is often crucial for the clinician to make an appropriate diagnosis and monitoring of shock. For example, very cold skin temperature will independently prolong CRT (an acceptable false positive of CRT measurement). For each time interval, the device will measure and monitor both CRT and skin temperature.

When a medical treatment is administered to the patient, the CRT may be measured thereafter on a periodic basis, and pulse pressure and/or blood oxygenation and/or PU may be measured continuously or periodically, but typically at smaller intervals than CRT. If the patient's reaction to the given treatment is positive, then in time the CRT will be reduced, indicating a significant improvement in the physiological condition of the patient until the CRT value goes below the safe 2 seconds level.

When measuring CRT it is essential that pressure be applied only to capillary vessels while maintaining normal blood flow. In some embodiments of a system in accordance with the invention, a programmable mechanical unit applies an accurate measurable amount of pressure to the skin.

In some embodiments, one said cardio-respiratory sensors comprises a blood oxygenation (BO) sensor module configured for monitoring blood oxygenation state, wherein operation of said BO sensor module is based on pulse oximetry techniques. The BO sensor module may be adapted for measuring SpO2 and may optionally comprise at least one emitter for emitting red light and infra red light, and at least one photodetector for receiving backscattered light from a target area of said patient at said anatomical part. The at least one photodetector may be adapted for operating according to a transmission method, and wherein said at least one emitter and said at least one photodetector are in opposed relationship with respect to an extremity during operation of said apparatus. Alternatively, the at least one photodetector is adapted for operating according to a reflectance method, and wherein said at least one emitter and said at least one photodetector are in adjacent relationship.

In some embodiments, one said cardio-respiratory sensors comprises a peripheral perfusion (PU) sensor module configured for monitoring a peripheral perfusion parameter other than CRT. Optionally, operation of said PU sensor module is based on photoplethysmographic techniques and said PU sensor module comprises at least one emitter for emitting light in the visible or non visible spectrum, and at least one photodetector for receiving backscattered light from a target area of said patient. Alternatively, operation of said PU sensor module is based on vascular ultrasonography techniques, and said PU sensor module comprises at least one transducer for generating suitable ultrasonic waves, and at least one transducer for receiving sound waves reflected from a target area of said patient. Alternatively, operation of said PU sensor module is based on laser Doppler flowmetry techniques and said PU sensor module comprises at least one optic fiber operatively connected to a laser for emitting light, and at least one optical fiber for receiving backscattered light from a target area of said patient. Alternatively, operation of said PU sensor module is based on suitable plethysmographic techniques.

In some embodiments, one said cardio-respiratory sensors comprises a blood pressure (BP) sensor module configured for monitoring at least one of blood pressure, pulse rate, systemic vascular resistance. In one embodiment, the BP sensor module is based on suitable Penaz techniques. Optionally, the BP sensor module comprises a plethysmograph and a pressure cuff, wherein a pressure applied by the cuff is controllable using an output of said plethysmograph such as to maintain the output from the plethysmograph substantially constant.

Optionally, the apparatus further comprises a body temperature sensor for measuring a body temperature of said patient at said same anatomical part. The apparatus may comprise a suitable data interface adapted for operative connection to an external control and data storage apparatus.

In one embodiment, the first cardio-respiratory sensor module comprises said CRT sensor module configured for monitoring a capillary refill time (CRT), and said second cardio-respiratory sensor module comprises said blood oxygenation (BO) sensor module configured for monitoring blood oxygenation state.

In another embodiment, said first cardio-respiratory sensor module comprises said CRT sensor module configured for monitoring a capillary refill time (CRT), and said second cardio-respiratory sensor module comprises said blood pressure (BP) sensor module configured for monitoring at least one of blood pressure, pulse rate, systemic vascular resistance.

In another embodiment, said first cardio-respiratory sensor module comprises said comprises said blood oxygenation (BO) sensor module configured for monitoring blood oxygenation state, and said second cardio-respiratory sensor module comprises said blood pressure (BP) sensor module configured for monitoring at least one of blood pressure, pulse rate, systemic vascular resistance.

In another embodiment, the first cardio-respiratory sensor module comprises said PU sensor module configured for monitoring a perfusion parameter other than capillary refill time (CRT), and said second cardio-respiratory sensor module comprises said blood oxygenation (BO) sensor module configured for monitoring blood oxygenation state.

In another embodiment, the first cardio-respiratory sensor module comprises said PU sensor module configured for monitoring a perfusion parameter other than capillary refill time (CRT), and said second cardio-respiratory sensor module comprises said blood pressure (BP) sensor module configured for monitoring at least one of blood pressure, pulse rate, systemic vascular resistance.

In another embodiment, the first cardio-respiratory sensor module comprises said CRT sensor module configured for monitoring a capillary refill time (CRT), and said second cardio-respiratory sensor module comprises said PU sensor module configured for monitoring a perfusion parameter other than capillary refill time (CRT).

In another embodiment, the first cardio-respiratory sensor module comprises said CRT sensor module configured for monitoring a capillary refill time (CRT), said second cardio-respiratory sensor module comprises said blood oxygenation (BO) sensor module configured for monitoring blood oxygenation state; and said third cardio-respiratory sensor module comprises said blood pressure (BP) sensor module configured for monitoring at least one of blood pressure, pulse rate, systemic vascular resistance.

In another embodiment, the first cardio-respiratory sensor module comprises said CRT sensor module configured for monitoring a capillary refill time (CRT), said second cardio-respiratory sensor module comprises said blood oxygenation (BO) sensor module configured for monitoring blood oxygenation state; and said third cardio-respiratory sensor module comprises said PU sensor module configured for monitoring a perfusion parameter other than capillary refill time (CRT).

In another embodiment, the first cardio-respiratory sensor module comprises said CRT sensor module configured for monitoring a capillary refill time (CRT), said second cardio-respiratory sensor module comprises said blood pressure (BP) sensor module configured for monitoring at least one of blood pressure, pulse rate, systemic vascular resistance, and third cardio-respiratory sensor module comprises said PU sensor module configured for monitoring a perfusion parameter other than capillary refill time (CRT).

In another embodiment, the first cardio-respiratory sensor module comprises said blood oxygenation (BO) sensor module configured for monitoring blood oxygenation state; said second cardio-respiratory sensor module comprises said PU sensor module configured for monitoring a perfusion parameter other than capillary refill time (CRT), and said third cardio-respiratory sensor module comprises said blood pressure (BP) sensor module configured for monitoring at least one of blood pressure, pulse rate, systemic vascular resistance.

In another embodiment, the first cardio-respiratory sensor module comprises said CRT sensor module configured for monitoring a capillary refill time (CRT), said second cardio-respiratory sensor module comprises said blood oxygenation (BO) sensor module configured for monitoring blood oxygenation state; said third cardio-respiratory sensor module comprises said blood pressure (BP) sensor module configured for monitoring at least one of blood pressure, pulse rate, systemic vascular resistance, and said fourth cardio-respiratory sensor module comprises said PU sensor module configured for monitoring a perfusion parameter other than capillary refill time (CRT).

The sensing device may be operatively connected to the user interface via a suitable cable, or via a suitable wireless connection, such as infrared, laser or other optical transmission, or radio frequency (RF) communication, for example, or in any other suitable manner.

The sensing device may be operatively connected to a remote user interface via any method of transmission, such as for example Telephony, Internet, RF, optical connection, etc.

Optionally, the apparatus may be adapted for accommodating a finger of said patient, the finger comprising said same anatomical part. The apparatus may comprise a lumen for accommodating said finger such that each said cardio-respiratory sensor can measure its corresponding said cardio-respiratory parameter at said same anatomical part. The apparatus may further comprise a sheath adapted to be worn over said finger, wherein said lumen is adapted to accommodate said finger having said sheath worn thereon. The sheath, which is per se novel, may comprise at least one optical portal comprising at least one of an aperture and an optical transparent window for allowing mechanical and optical communication, respectively, between an inside and an outside of the sheath. The at least one of an aperture and an optical transparent window may be positioned such as to provide registry with said cardio-respiratory sensors when said sheath is inserted within said lumen.

The sheath may be made from a disposable material. In one embodiment, the sheath comprises an upper portion foldable over a lower portion in overlying relationship by means of a deformable first end portion therebetween, such as to define an opening at a second end thereof opposed to said first end, and an inner space for accommodating a finger. Further, the sheath may be adapted for becoming unusable as a sheath after being removed from a finger.

The present invention is also directed to a system for providing data indicative of cardio-respiratory state of a patient comprising: an apparatus according to the invention as defined herein; and a user interface for enabling data relating to at least two said cardio-vascular parameters obtained from said apparatus to be at least one of processed and displayed.

The interface may be adapted for displaying said data for at least one time window comprising an elapsed time starting at or after commencement of operation of said system with respect to said patient. The user interface may be adapted for enabling at least two said cardio-respiratory parameter data with respect to elapsed time to be scrolled to enable any time window comprising such data to be displayed. The data may be displayed at least one of graphically and as alphanumeric characters. The user interface may comprise a suitable screen display. The apparatus may be operatively connected to said user interface via at least one of a suitable cable and a suitable wireless connection, for example. The wireless connection may be via the Internet, for example. Optionally, the apparatus may be integrated with said user interface in the form of a handheld device.

The present invention is also directed to a method for providing data indicative of cardio-respiratory state of a patient comprising measuring at least two cardio-respiratory parameters of said patient, wherein said at least two cardio-respiratory parameters are different one from the other and are measured at a same anatomical part of said patient. Optionally, the method may comprise measuring at least three cardio-respiratory parameters of said patient, wherein said at least three cardio-respiratory parameter are different one from the other and are measured at a same anatomical part of said patient. Optionally, the method may comprise measuring at least four cardio-respiratory parameters of said patient, wherein said at least four cardio-respiratory parameters are different one from the other and are measured at a same anatomical part of said patient.

One said cardio-respiratory parameter is blood oxygenation state; measurement of said blood oxygenation state may be based on pulse oximetry techniques. Another said cardio-respiratory parameter may be capillary refill time (CRT). Measurement of said CRT may comprise the steps of: acquiring an image of skin area to be gauged for a second wavelength illuminated with a light of a first wavelength from a light source to obtain a base-line color measurement, and determining the filling time of blood vessels in said area by comparison of the wavelength of at least one more additional images of the gauged skin area with said base-line color measurement.

The method may comprise the steps of:

(i) positioning image acquisition means so that an area of the skin lies substantially within the focal plane thereof;

(ii) illuminating said area having an original color with light radiation from said light source at said first wavelength at a level enabling said image acquisition means to discriminate between wavelengths;

(iii) acquiring an image of said area with said image acquisition means;

(iv) deriving a signal from said image, said signal representative of the wavelength of light originating from said area;

(v) storing the value of said signal which corresponding to said original color;

(vi) applying pressure on said area, said pressure having a magnitude and duration sufficient to expel blood out from said blood vessels, and for obtaining a signal having a value which corresponds to the maximum whitening of said area;

(vii) measuring the filling time by rapidly releasing said pressure and subsequently measuring and displaying the total period of time from maximum whitening until the value of said signal is substantially the same as said stored value.

The method may further comprise:
repeating the measurement of the filling time at different time intervals;
storing the values of all measurements; and
displaying a graphical representation of the measured filling times as a function of time, thereby obtaining a derivative of the capillary filling time on time d[CRT]/d[t], said derivative being an indication related to deterioration in the patient's physiological condition, or to the recovery of the patient from physiological distress.

The signal may be based on a portion of said area of skin close to but not including the part of the skin that is directly pressured. The method may further comprise the step of correcting said signal to compensate for effects that may be caused by skin movement after said releasing of pressure. The correction may be performed using a suitable algorithm. The method may comprise the step of determining parameters including skin resistance to pressure as a function of depression of the skin responsive to the pressing, and providing said parameters as inputs to said algorithm. The method may further comprise the step of measuring a first skin temperature of a second skin area close to said first mentioned area, wherein said second skin area is substantially unaffected by heat effects generated by said apparatus. The method may further comprise the step of measuring a second skin temperature of said first mentioned area, wherein said first mentioned skin area is substantially unaffected by heat effects generated by said apparatus. The method may further include the step of modifying the filing time in step (vii) according to the magnitude of at least one of said first temperature or said second temperature. The CRT data may be obtained from a target area on a finger. Yet another said cardio-respiratory parameter may be a perfusion parameter (PU) other than capillary refill time (CRT). Measurement of said PU parameter may be based, for example, on any one of: photoplethysmographic techniques; vascular ultrasonography techniques; Doppler flowmetry techniques; suitable plethysmographic techniques. Another said cardio-respiratory sensors may be a blood pressure parameter including at least one of blood pressure, pulse rate, systemic vascular resistance. Measurement of said blood pressure parameter may be based on any suitable Penaz techniques.

Optionally, data obtained for said at least two cardio-respiratory parameter and/or a body temperature of the patient may be concurrently displayed. Optionally, at least two said cardio-respiratory parameters are monitored over a period of time. Optionally, data obtained for said at least two cardio-respiratory parameters with respect to elapsed time may be scrolled to enable any time window within said period of time comprising such data to be displayed. Optionally, data obtained for said at least two cardio-respiratory parameters may be displayed at least one of graphically and as alphanumeric characters.

The said at least two cardio-respiratory parameters may be measured at substantially the skin portion or same extremity. Other said cardiovascular parameters may be measured or monitored at the same extremity or skin portion, or at a different extremity or skin portion. For example, the extremity may be a nose, ear, finger, hand, arm, toe, foot, leg.

In some applications, the method of the invention is particularly for the diagnosis of any one of shock, early shock and dehydration.

Thus, the apparatus, system and method of the invention allows for often immediate diagnosis of the cardiorespiratory state of a patient, often including the state of shock or dehydration of a patient, and allows better monitoring of cardio-respiratory parameters such as for example, PU, SpO2, PI, blood pressure and so on in the same region as the CRT measurement for any desired diagnostic purpose, such as regarding shock, organ or skin transplants, diabetes, drug interactions, and others which have an effect in the cardio-respiratory process.

By means of the present invention, it may be possible to make, even in a pre-hospital setting, an early diagnosis of cardiovascular and respiratory state of a patient, and also of shock, as well as enabling the determination of whether the drug being administered to a patient in shock is having the desired therapeutic effect.

A feature of measuring CRT together with other cardio-respiratory parameters using sensing integrated instrumentation according to the invention is that it enables early detection of a shock syndrome (compensated shock, prior to the reduction of blood pressure) and indicates its severity. This makes possible prompt treatment of patients who can then survive a shock-related condition which may be fatal if untreated or if treated too late.

This makes possible prompt treatment of patients who can then survive a shock-related or other cardiorespiratory based or related condition, which may be fatal if untreated or if treated too late. In addition, the invention enables the monitoring of changes in capillary flow in skin areas of peripheral body organs. This provides a rapid yet accurate reading of the patient's condition, making it possible to treat the patient without delay to avoid damaging consequences.

Some shock-related conditions are related to inadequate flow in a specific organ. These medical conditions are common in patients after orthopedic surgery, flap reconstruction surgery, or patients who suffer from a severe peripheral vascular disease. By being highly sensitive to changes in capillary flow, a system in accordance with the invention is applicable to these medical shock-related conditions.

The sensing apparatus for measuring cardio-respiratory parameters may also be coupled to other sites in the patient's body that are rich in subcutaneous blood vessels, such as to the lip or to the ear lobe.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1(a), 1(b) and 1(c) illustrate variations of the elements of an embodiment of the system of the invention.

FIG. 2 schematically illustrates in cross sectional view elements of a first embodiment of sensing apparatus that may be comprised in the system of FIG. 1.

FIG. 3 illustrates an embodiment of a CRT sensor module of the sensing apparatus of FIG. 2.

FIG. 4 is a block diagram showing elements of the display apparatus and of the display and processor unit included in the embodiment of FIG. 1.

FIG. 5 is a block diagram showing elements of the display apparatus and of the display and processor unit included in a variation of the embodiment of FIG. 1.

FIG. 6 illustrates an example of a display format for the display and processor unit of a variation of the embodiment of FIG. 1.

FIG. 7 illustrates a variation of the embodiment of the CRT sensor module of FIG. 2.

FIG. 8 illustrates in fragmented isometric view the locking means of the sheath of the sensing device of FIG. 2.

FIG. 9 illustrates in partial cross-sectional view the locking means of FIG. 8.

FIG. 10 is a graph showing an effect of skin temperature on CRT readings.

FIG. 11 is a graphical representation of the CRT data that may be obtained with the embodiment of FIG. 1.

FIG. 12 is a graphical representation of CRT, as a function of the level of shock, for obtaining inferences related to the trend of the patient's physiological condition in reaction to medical treatment.

FIG. 13 is a graphical representation of an example of PU data and CRT data that may be obtained with some embodiments of the present invention.

FIG. 14 illustrates in cross sectional view a variation of the embodiment of FIG. 2

FIG. 15 schematically illustrates in cross sectional view elements of a second embodiment of sensing apparatus that may be comprised in the system of FIG. 1.

FIG. 16 schematically illustrates in cross sectional view elements of a third embodiment of sensing apparatus that may be comprised in the system of FIG. 1.

### DETAILED DESCRIPTION

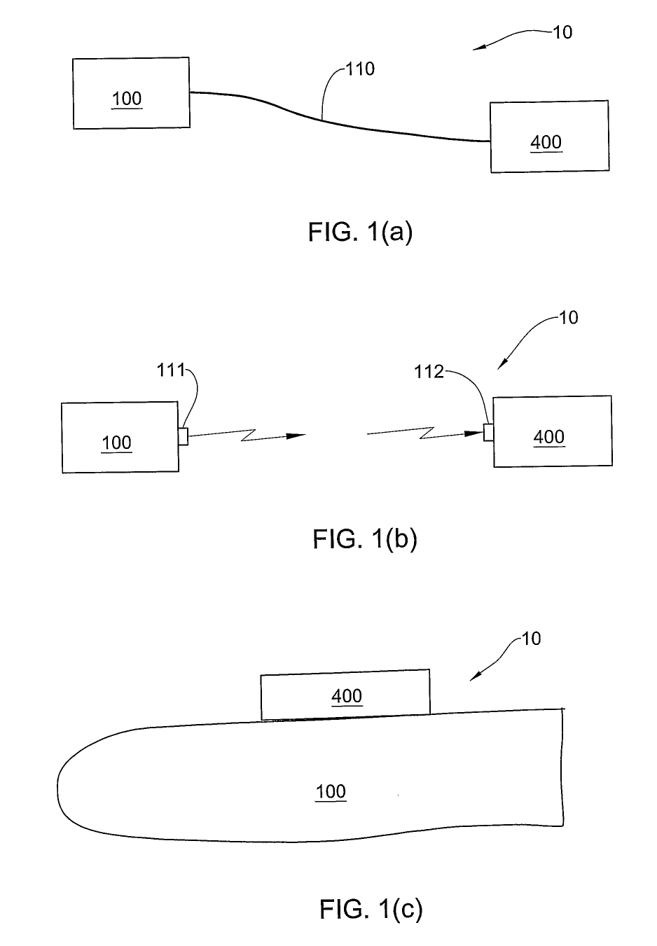

A first embodiment of the system of the present invention is illustrated in FIG. 1(a), and is generally designated with the numeral 10. The system 10 comprises a sensing apparatus 100, operatively connected to a user interface in the form of the processing and display unit 400, via a cord 110 through which data obtained by the sensing apparatus 100 is fed for processing and display, and optionally commands are transmitted to the apparatus 100 by the unit 400. For example, the cord may be a fiber optic cable, a bus or an electrical cable. Alternatively, and as illustrated in FIG. 1(b), the cable may be replaced or supplemented with a wireless transmitter and receiver system, 111, 112, in the apparatus 100 and the unit 400 for exchanging data and commands between the two elements of the system. Such a transmitter and receiver system may be infra-red based, or radio based for example, or may make use of any other suitable transmitting and receiving technique. The processing and display unit 400 may be, for example, a personal computer that uses control and processing software to process the data received from the sensing apparatus 100.

Alternatively, the system 10 may be in the form of an integral device, such as for example a hand-held device, wherein the various elements thereof, are integrated within a common housing. The device may be configured to be compact and portable, for example, and thus be suitable for home use, hospital use, and also for use with ambulance and paramedic teams, for example.

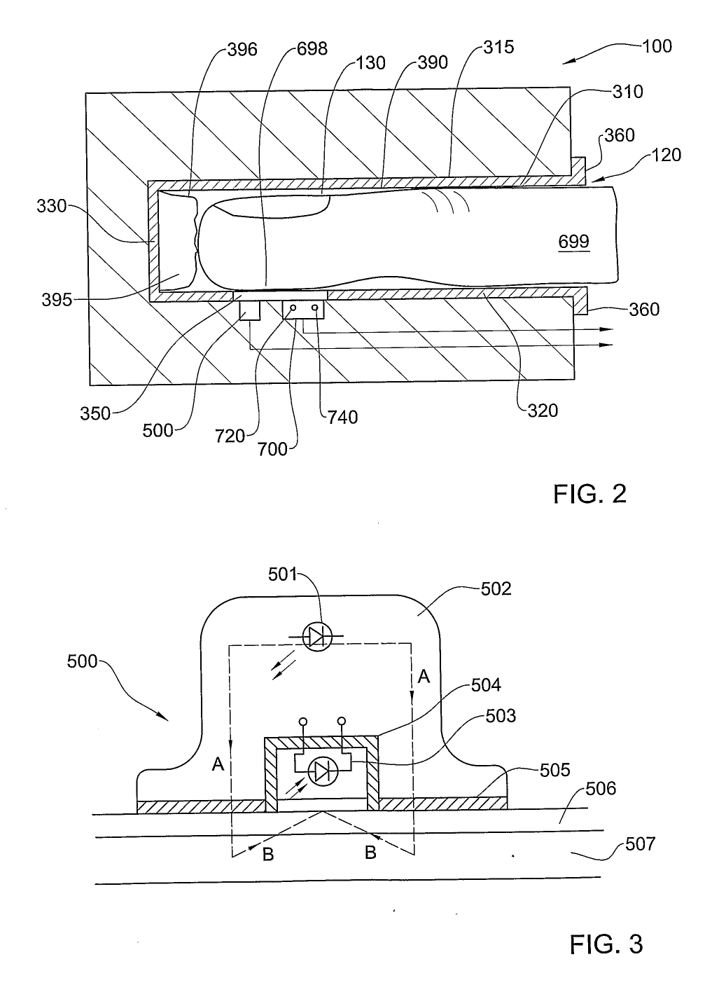

Referring to FIGS. 2 and 14, the sensing apparatus 100 is adapted for providing CRT data as well as blood oxygenation data from the same general anatomical area of the body. In this embodiment, such data may be obtained from an extremity, such as a finger 699, for example, though the apparatus may be adapted for providing the required cardio-respiratory parameters from any other extremity, mutatis mutandis. Thus, the sensing apparatus 100 comprises a finger receiving opening 120, and a lumen 130 for accommodating a patient's finger 699 during operation of the system 100. The sensing device comprises a CRT sensor module 500 for providing CRT data, and a blood oxygenation sensor module 700 for providing blood oxygenation data with respect to the same general vascular bed of the patient.

A number of embodiments for the CRT sensor module 500 will now be described.

FIG. 3 schematically illustrates the structure of a CRT sensor module 500 according to one embodiment thereof, for example as disclosed in U.S. Pat. No. 6,685,635, also assigned to the present assignee, and the contents of which are incorporated herein in their entirety. Module 500 is provided for obtaining CRT data, and includes a continuous (non-modulated) or a pulsating (modulated) light source 501, such as a Light Emitting Diode (LED) driven by a rectangular voltage pulse generator at a predetermined frequency fₒ. Light source 501 is enclosed in a light-reflecting external housing 502 having an opening in its bottom side so that most of the light radiation emitted from light source 501 is directed toward the bottom side in one direction “A”. External housing 502 has within it an opaque internal housing 504 containing a light sensor 503, such as a photodiode, a phototransistor, a photo-resistor or a photoelectric cell. Internal housing 504 has an opening in its bottom side which permits light rays to enter therein only through its bottom side. The bottom sides of external housing 502 and internal housing 504 are aligned with each other and are covered by a transparent rigid layer 505. This layer serves to apply pressure on the skin while enabling light to pass therethrough in both directions.

Transparent rigid layer 505 of module 500 is pressed into contact with the exterior layer 506 of the skin. Pressure is applied automatically on the external housing 502 toward the skin surface in a perpendicular direction by means of a suitable actuator (not shown). The external housing delivers the pressure to the transparent rigid layer 505 which transfers it through exterior layer 506 to the interior layer 507 of the skin containing most of the subcutaneous blood vessels (capillaries).

As a result, when the magnitude of the applied pressure is adequate and is maintained for sufficient period of time, blood is then forced out of the pressurized capillaries and the color of the interior layer 507 of skin becomes much brighter (i.e. substantially white). Light rays emitted from light source 501 penetrate into the skin into this layer 507 and are partially reflected back in direction “B”, into the internal housing 504. The degree of reflection from interior layer 507 is inversely related to blood flow in the capillaries under pressure inasmuch as blood absorbs light, the more blood in the capillaries the lesser is the reflected light.

The reflected light is aggregated by light sensor 503 which yields an electric signal whose magnitude depends on the instantaneous color of the skin. Under zero pressure (i.e., full blood flow), the skin color is normally pink and therefore less light is reflected back from the capillaries. When the skin is subjected to pressure and blood is expelled from the capillaries, the skin color is then white. Hence when the skin is pink, the intensity of reflected light is relatively low and when the skin is white the intensity of reflected light is significantly higher. Consequently, changes in magnitude of the electric signal produced by light sensor 503 afford an accurate measure of the capillary filling time and rate. The module 500 is connected to a pulsed power supply for energizing light source 501 and for operating data collection, processing and display circuitry to process the signals yielded by light sensor 503 and for displaying the measurement results.

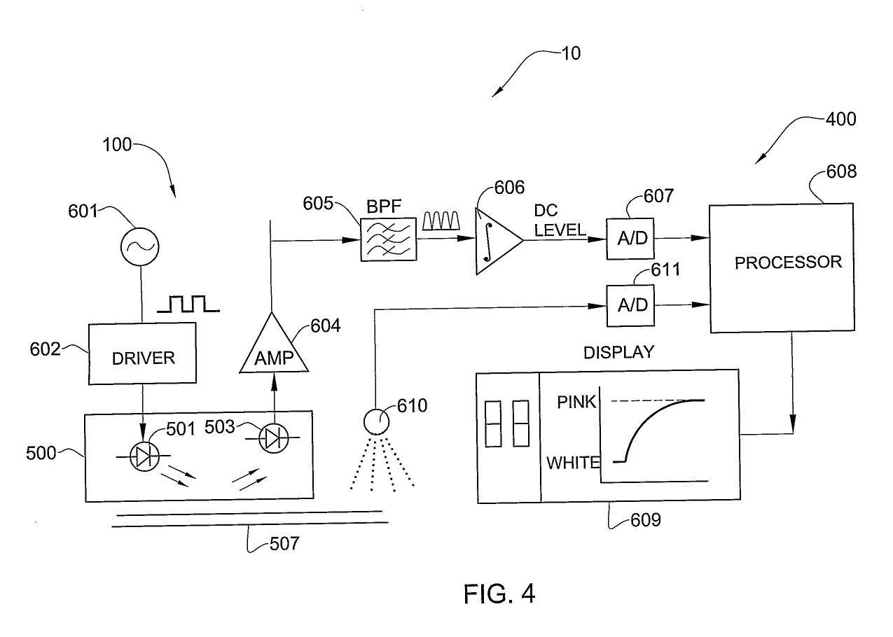

As illustrated in FIG. 4, in one embodiment of the system 10, the processing and display unit 400 comprises a rectangular pulse oscillator 601 operated at a suitable frequency, for example fₒ=18 KHz. The output of oscillator 601 is fed into a driver 602 which provides rectangular output pulses having sufficient energy to power light source 501 to emit light pulses at the same frequency fₒ. Light reflected from the skin is converted by light sensor 503 to a corresponding pulsatory electrical signal. This signal is fed into an amplifier 604 operating within a frequency band that includes frequency fₒ to increase the amplitude of the electrical signal. Alternatively, oscillator 601 and driver 602 may be comprised in the apparatus 100 or in an auxiliary apparatus operatively connected thereto.

Light sensor 503, included in module 500, may be sensitive to the full color spectrum, the visible spectrum or beyond the same, for example infrared, or alternatively most sensitive to light radiation to a particular range of wavelengths, for example between red and infra-red in the color spectrum; to a particular range of wavelengths, for example between red and blue, for example green; for example also to background light sources, such as external light radiation which adds an unwanted 50/60 Hz signal, or to sunlight which adds an unwanted DC level. Therefore the electrical output signal includes interfering components as well as the desired component at frequency fₒ. The interfering components are reduced in magnitude by the amplifier 604 which is tuned to amplify the desired component at frequency fₒ to a greater degree than the unwanted components.

The amplified electrical signal from amplifier 604 is further filtered by a Band-Pass-Filter (BPF) 605. This filter is tuned to pass only the desired component at frequency fₒ and to reject all other unwanted components. BPF 605 may be implemented as an active filter using Integrated Circuit (IC) technology. The resultant filtered signal at the output of BPF 605 is a rectified sine wave which is fed into an integrator circuit 606. Integrator circuit 606 outputs a Direct Current (DC) level proportional to the magnitude of the rectified sine wave and hence the magnitude of light reflected from the skin. It is therefore highly sensitive to changes in skin color.

The DC signal is fed into an Analog to Digital Converter (ADC) 607, which converts the DC level into a corresponding digital word. The digital data is fed into a digital processor 608 which analyzes the data and display the results on a suitable display 609. Display 608 exhibits a digital value representing the measurement results (i.e., the CRT), and a graphical representation of the measurement process as a function of time. The graphical representation provides an indication of whether or not the measurement results are reasonable, and if desired, the measurement can be repeated. Other data processed results, such as statistical data, can be also displayed to provide indications related to the reaction of the patient to medical treatment.

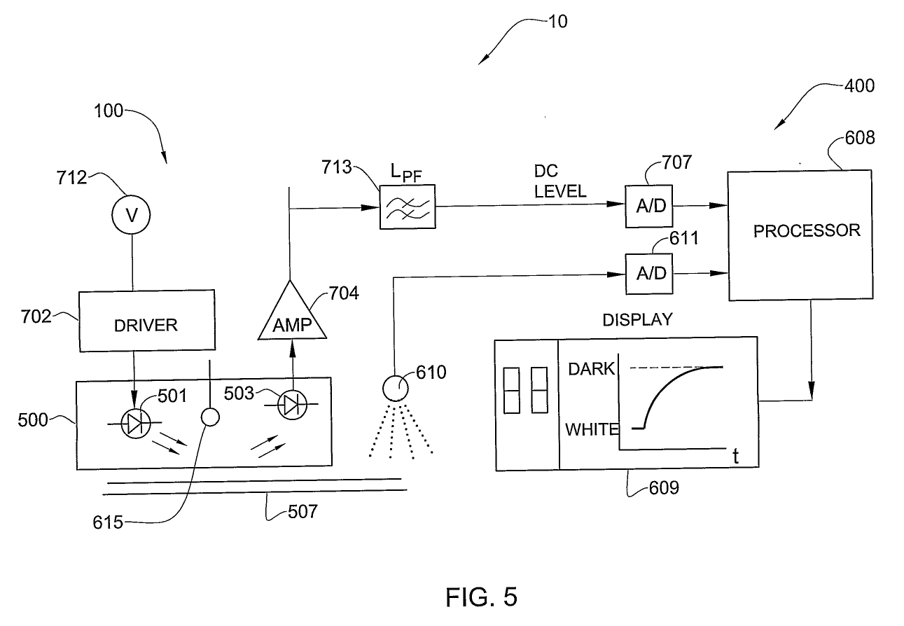

Alternatively, and as illustrated in FIG. 5, one embodiment of the processing and display unit 400 comprises a constant source 712 operated at a DC voltage. The output of source 712 is fed into a driver 702 which provides energy to power light source 501 to emit non-modulated, continuous light. Light reflected from the skin is converted by light sensor 503 to a corresponding electrical signal. This signal is fed into an amplifier 704 operating at near-DC frequency band to increase the amplitude of the electrical signal.

Light sensor 503 is may be sensitive to the full color spectrum, the visible spectrum or beyond the same, for example infrared, or alternatively most sensitive to light radiation to a particular range of wavelengths, for example between red and infra-red in the color spectrum, or for example between red and blue, for example green. Thus, the description above relating to skin color is understood to also refer to the wavelength measured at the skin site, originating at the surface of the skin or below the same time, according to the penetration of the illuminating wavelength, mutatis mutandis. The sensor 503 may also be sensitive to background light sources, such as external light radiation which may add an unwanted 50/60 Hz signal, or to sunlight which adds an unwanted DC level. Therefore the electrical output signal may include interfering components as well as the desired DC level. The interfering components are reduced in magnitude by the amplifier 704 which is tuned to amplify the desired DC signal to a greater degree than the unwanted components.

The amplified electrical signal from amplifier 704 is further filtered by a Low-Pass-Filter (LPF) 713. This filter is tuned to pass only the desired component of low signal frequencies and to reject all other unwanted components. LPF 713 is implemented as an active filter using Integrated Circuit (IC) technology. The resultant filtered signal at the output of LPF 713 is a direct current (DC) level proportional to the magnitude of the light reflected from the skin. It is therefore highly sensitive to changes in skin color.

Alternatively, source 701 and driver 702 may be comprised in the apparatus 100 or in an auxiliary apparatus operatively connected thereto.

The DC signal is fed into an Analog to Digital Converter (ADC) 707, which converts the DC level into a corresponding digital word. The digital data is fed into a digital processor 608 which analyzes the data and display the results on a suitable display 609. Display 609 exhibits a digital value representing the measurement results (i.e., the CRT), and a graphical representation of the measurement process as a function of time, as is further described herein. The graphical representation provides an indication of whether or not the measurement results are reasonable, and if desired, the measurement can be repeated. Other data processed results, such as statistical data, can be also displayed to provide indications related to the reaction of the patient to medical treatment.

Alternatively, other arrangements may be used for the CRT sensor module 500, and for processing and displaying the CRT data via unit 400, for example as described in U.S. Pat. No. 6,685,635.

The signal representative of changes in skin coloring (i.e., reflected wavelength from the skin area being tested, in the visible or invisible wavelengths) can also be affected by optical amplitude variations, which may be caused at times by the movement of skin back to its original position after the pressure is released by the sensor, for example. In order to correct for this effect, the processing procedure for the signals may be modified to include a compensating algorithm that may be applied before the computation of CRT time.

For embodiments of the CRT sensor module 500 where the distance between the color or light sensor and skin is small, the depression of the skin under the action of the mechanical pressure inducer (e.g. a plunger) may have an influence on the intensity of light finally reaching the sensor. This is so when the amplitude of the skin depression is not insignificant with respect to the color or light sensor-to-skin distance. When the mechanical pressure inducer is at maximum depth with respect to the skin or tissue, the distance to the sensor is greater, and thus intensity of the light received by the sensor is lower, in line with the inverse square law. When the skin springs back, after the mechanical pressure is released, i.e., at the beginning of the measurements for CRT, the distance progressively reduces, and the intensity progressively increases. Thus a positive intensity effect occurs during the monitoring of the skin color or light intensity after blanching due to the skin returning to its original position. At the same time, there also occurs a negative intensity effect, i.e. a falling in the intensity measured by the color sensor, due to the color of the skin changing from white to pink. While the sensor senses the combined effect of positive and negative effect, it is only the negative effect due to CRT that is of interest. According to another aspect of the present invention, the intensity effects due to distance may be corrected or eliminated at source to obtain the true changes in intensity due to changes in color.

In some embodiments of the CRT sensor module 500, the intensity effects due to changes in distance may be compensated by first determining the spring-back properties of the skin when the mechanical pressure is released. Knowledge of these properties enables the changes in distance with respect to time for the skin to be calculated during the restoration period, as the skin returns to the original position. The variation of distance with time can in turn be converted into relative changes in intensity, since the intensity obeys an inverse square law with respect to distance. The relative changes in intensity can then be related to a baseline intensity value, such as the original intensity that is recorded just after the mechanical pressure is released, for example. Alternatively, the baseline intensity may be the original intensity of the illuminating radiation, i.e., the intensity at the source, in which case the intensity is inversely proportional to a 4^{th} power of the distance. These spring-back properties of the skin may change from patient to patient, and from apparatus to apparatus, and may also vary even with the same patient, form example depending on the degree of hydration of the patient.

Considering the skin (or other tissue) to behave as a simple spring, the resistance of the skin to deformation by the mechanical pressure inducer may be assumed to be in some way proportional to the depth of the pressure inducer with respect to the skin. Suitable stress or strain measurement means may be provided, together with displacement measurement means, and thus the spring constant (which may actually vary with depth) of the skin under the particular conditions of the current CRT test may be obtained. Once the inducer is released from the skin, a suitable algorithm can estimate the trajectory of the skin back to the original position using the established spring constant, and thus the changes in distance with time for the skin can be converted to an intensity effect. This intensity effect may then be subtracted from the actual intensity recorded via the color or light sensor to provide a corrected intensity value for the light received from the skin or tissue being tested which is indicative of CRT effects.

In a variation of the embodiment of FIG. 3, the distance between the skin or tissue being tested and the color or light sensor is kept constant during capillary filing, such that no substantial spring-back occurs, and thus CRT sensor module 500 is replaced with of the CRT sensor module 580 that is similar to of the CRT sensor module 500 as described herein, mutatis mutandis, but is further configured to maintain this distance constant. Referring to FIG. 7, for example, the CRT sensor module 580 may comprise a guard 810 in the form of a ring 815 that is spaced from the body 850 of the device via struts 820. A mechanical plunger 830 moves from a retracted position, displaced from the ring 815, to a deployed position just below the level of the ring such as to provide pressure to the skin. As the plunger is retracted, the pressure is released from the skin but this is prevented from springing back due to the ring. The body 850 houses the color or light sensor (not shown), as well as other components such as illumination means, for example, in a similar manner to that described for the CRT sensor module 500, mutatis mutandis.

For more accurate CRT readings it may be necessary to measure the skin surface temperature and record it prior to each CRT measurement.

Referring to FIGS. 14, 4 and 5, in order to factor into the processing of the reflected light intensity the influence thereon of skin temperature, the CRT sensor module 500, (or CRT sensor module 580, mutatis mutandis) comprises a heat sensor 610, such as an infrared detector or a thermistor, whose output signal varies in magnitude as a function of the intensity of infrared rays emanating from the skin surface in the course of CRT diagnosis. Infrared detector 610 is responsive only to the heat of the skin, not to light reflected from the skin surface.

The electrical signal yielded by heat sensor 610 is not pulsed and has a magnitude which is a function of skin temperature. This signal is digitized in an A/D converter 611 whose digital output is entered into computer microprocessor 608. Microprocessor 608 is programmed by software to factor into the CRT reading the effect thereon of skin temperature. This corrected reading is of value in real time diagnosis of a patient's shock-related state, for it takes into account the skin temperature of the patient when in shock. It is of somewhat lesser value when monitoring the condition of a patient being treated for shock.

One form of skin temperature sensor may be a thermometer which can be placed directly on the skin surface of a patient being diagnosed for shock, to provide an electrical signal whose magnitude depends on the existing skin temperature. The thermometer signal is entered into microprocessor 608 into which is also entered the CRT signal indicative in terms of seconds, the shock state of the patient.

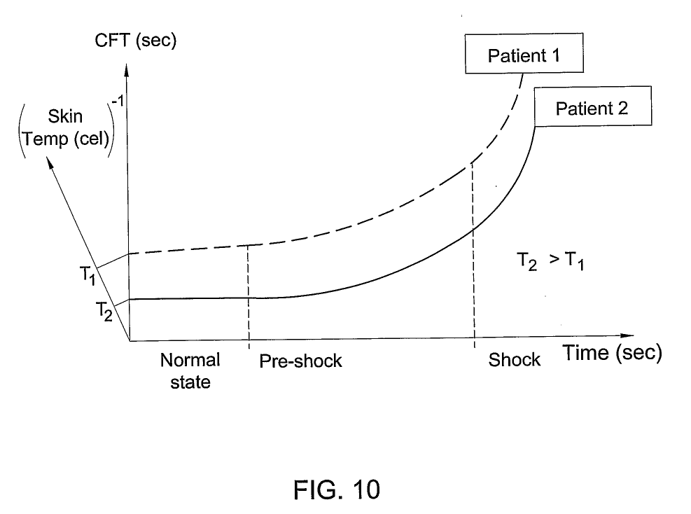

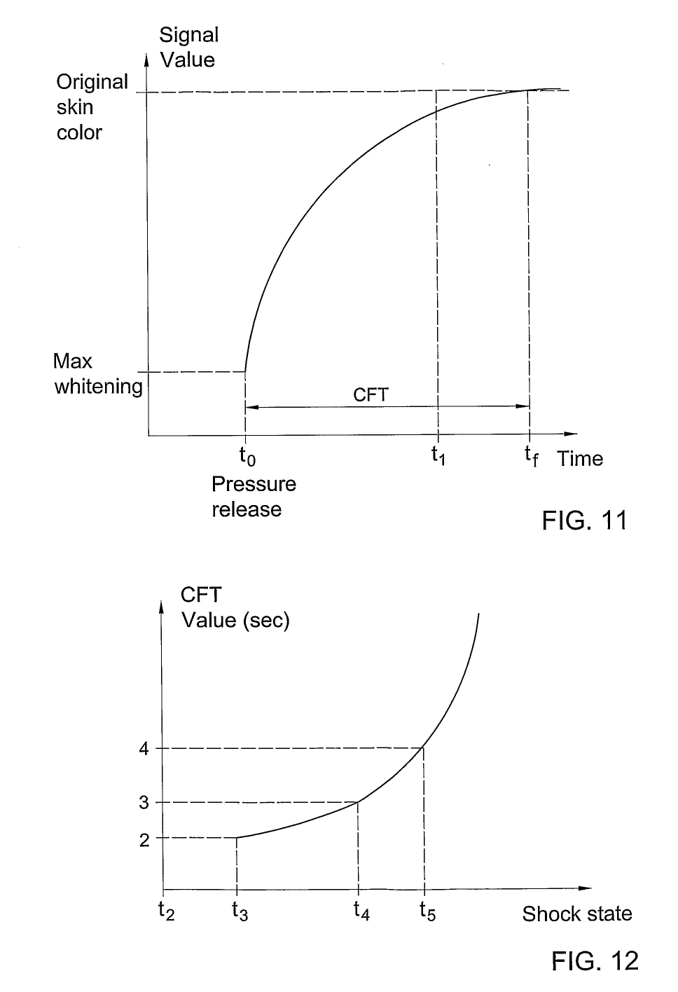

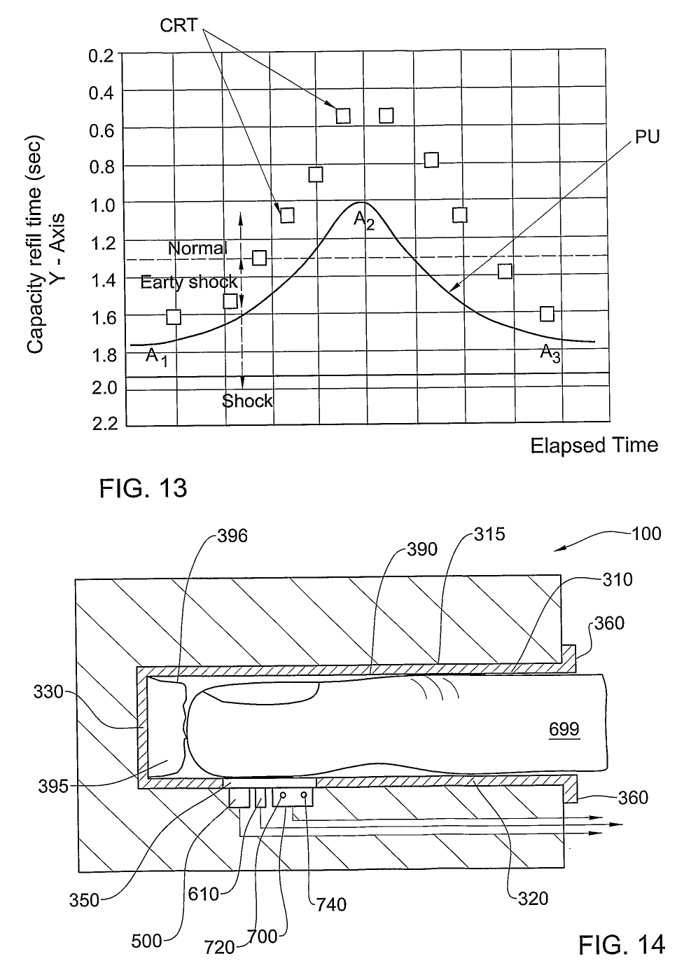

FIG. 10 illustrates the effect of skin temperature on CRT readings for patients 1 and 2 having different skin temperatures T1 and T2, where T2 is greater than T1. It will be seen that in a normal no-shock state, the CRT readings which indicate this state in terms of seconds are different, thereby reflecting the effect on the CRT readings of the degree of difference between temperatures T1 and T2. Similar differences appear for the pre-shock and shock states.

As has been described above, a temperature sensor may be used to determine skin temperature, which can then be used to correct the CRT for temperature effects.

It is to be noted that it is often desirable to determine the CRT of a patient at the actual skin temperature of the patient that is not influenced by the device of the invention itself. Typically, skin temperature should be a function of the internal perfusion effects in the skin. However, the closeness of the device, to the skin, particularly when taped thereto generates some local warmth, as the part of the skin covered by the device is now at least partially insulated from the outside environment. In addition, the illumination source itself can also generate some additional warmth to the skin, the temperature of which naturally increases. Preferably, and as illustrated in FIGS. 14, 4 and 5, a heat sensor 610 may be provided outside the main body of the CRT sensor module 500 and substantially beyond the influence of the illumination source or the main contact point between the device and the skin. This heat sensor thus provides a skin temperature Ta, and at the beginning of testing, the part of the skin being tested is at this temperature. As testing continues, this part of the skin gets progressively warmer, until steady state conditions are reached, wherein the temperature of this part of the skin reaches Tb, higher than Ta. At such conditions, the CRT determined with respect to the skin portion is thus associated with Tb rather than Ta, and needs to be corrected to Ta, which is more representative of the skin temperature minus the device temperature effects. According to this aspect of the invention, a second temperature sensor is provided for measuring the temperature of the skin, substantially similar to sensor 610 as described herein, mutatis mutandis, but such that it is influenced by the heating effects of the illumination means and the main contact points between the device and the skin. Thus, referring to FIG. 5, the second temperature sensor 615 may be located next to the light sensor 503 within internal housing 504, while the first sensor (not shown) may be provided outside of the external housing 502, but still within the device 500. According to this aspect of the invention, the temperatures Ta and Tb are measured via the first and second heat sensors, respectively, and suitable processing means monitors the changes in temperature as a function of time. At the beginning of testing, when Tb is increasing with respect to Ta, the CRT measurement may be adjusted according to temperature Ta. As the skin portion being monitored warms up due to the closeness of the probe, and due to heating from the light source, the CRT eventually corresponds to Tb, which is the temperature of the skin in the vicinity of the light source. At this point CRT needs to be adjusted to compensate for the increased temperature Tb. Between these two points in time, it is not straightforward to determine the actual temperature of the skin portion, in other words, how much of the skin (typically depth wise) is at Ta, and how much is at Tb. Accordingly, the processing means may provide, at least until steady state conditions are achieved, two values of CRT, one assuming that the tissue is at Ta, and the other correcting this CRT to Tb.

According to another aspect of the invention, measurement of the light intensity for CRT determination is carried out via the CRT sensor module on a skin or tissue portion that is close to but not directly acted upon by the mechanical pressure means. Repeated application of mechanical pressure to the same portion of skin can lead to some minor hemorrhaging of the capillaries in this area, which intensifies the red appearance of this portion. This has the effect of reducing the measured intensity value for the light received therefrom, and thus introduces an error in the determination of CRT. According to this aspect of the invention, the CRT sensor module 500 is adapted for enabling the light or color sensor to receive light reflected from the skin being tested, but not from the part of the skin within this portion that is actually being pressed by the mechanical pressure inducer. In one embodiment, the mechanical pressure inducer is in the form of a plunger, and the light sensor is located above the plunger. In this manner, the plunger itself prevents the part of the skin in contact with the plunger from being visible to the light sensor, which then receives light from the remainder of the skin portion. In another embodiment, the light intensities corresponding to the portion of skin under direct influence from the mechanical pressure inducer is electronically removed from the other light signals. In yet another embodiment of the CRT sensor module 500, suitable algorithms, embodied in the processing means, disregard all intensity measurements from a predetermined area of the sensor, corresponding to the area of skin that is subjected to mechanical pressure.

The CRT measurements can be carried out by other embodiments of the CRT sensor module 500 in a great variety of other ways, employing techniques which differ from those described herein, such as by using pneumatic apparatus for applying pressure to the patient's skin, or by using an Infra-Red camera rather than a video camera. Also one can store the history of CRT measurements of a patient and display the variation of the CRT curve with time.

The CRT data may be obtained from the measurements provided by the CRT sensor module 500 using any suitable algorithm, for example, as described in U.S. Pat. No. 6,685,635. Alternatively, another CRT computation algorithm may be used, based on principle of linear approximation of color (or other wavelength) recovering curve for each of the values, sampled during the color (or other wavelength) recovering process. A base point is defined for the color (or other wavelength) value, sampled from the blanched color (or other datum wavelength) stage of CRT test. For each other sample after this point, a line is constructed passing through the sample point and the base point. According to gradient of this line and to the base point, an approximation of CRT time (tᵢ) for the sampled value is calculated. The vector of approximated CRT values, (t₁, t₂, . . . tᵢ, . . . tₙ), is subjected to filtration, to remove incorrect as well as out-of-margins values. The filtration criteria and value margins depend on hardware parameters are applied. The, filtered CRT values are then manipulated to obtain the final CRT result, represented as the average, median or calculated by any other way from the filtered CRT values. Each action of the algorithm can be proceed during the sampling process or after the sampling process is done. A number of sample points may be analysed during each refill cycle of the capillaries.

Alternatively, the CRT computation algorithm is based on different slew rate analysis. For each pair of consecutive color sampling values during the color (or other wavelength) recovering stage, the increment (Ci) between the values is calculated. After the color (or other wavelength) recovering process is completed, there is thus provided a vector of the sampled increments, (C₁, C₂, . . . Cᵢ, . . . Cₙ). Each of increments represents the numerical derivation at the time point of the sample. The vector of numerical derivation values is subjected to filtration to remove incorrect as well as out-of-margins values. The filtration criteria and value margins depend on hardware parameters are applied. The filtered numerical derivation values are then manipulated to obtain the final derivation result, represented as the average, median or calculated by any other way, based on the recovering process derivation values. This value helps to calculate the simplified line of color recovering process and to define the CRT value, referenced to sampled value of maximal blanching and to the color (or other wavelength) value, sampled before the pressing and blanching the color (or other wavelength).

Alternatively, the vector of filtered numerical derivations is used for calculation of more sophisticated interpolating curve that assists to calculate the CRT value according to criteria from U.S. Pat. No. 6,685,635.

Each implementation of the above algorithms can occur during the sampling process or after the sampling process is done.

A number of embodiments for the blood oxygenation sensor module 700 will now be described.

Referring again to FIG. 2, in one embodiment, blood oxygenation sensor module 700 is based on pulse oximetry, and comprises a light emitter 720 having at least one red LED and at least one infrared LED, situated in the lumen 130 such that the light emitted from these emitters is reflected from a particular depth within the patient's finger 699, typically the subcutaneous tissue or deeper, and deeper than the capillaries of the dermis layer. A photodetector 740 may be provided in adjacent relationship to the emitter 720 and overlaying relationship with the measuring site, and the light from the emitter bounces to the detector across the site.

In an alternative embodiment (not illustrated), the blood oxygenation sensor module 700 is also based on pulse oximetry, but uses a transmission method rather than a reflectance method, and the light emitted from emitter 720 penetrates the patient's finger 699, and the emitter 720 and photodetector 740 are located generally opposed to each other in the lumen. for receiving the light that passes through the measuring site 698 of the patient's finger.

After the transmitted red (R) and infrared (IR) signals pass through the measuring site, or are bounced therefrom, and are received at the photodetector 740, the ratio of the intensities of the received red and infrared lights, R/IR, is calculated by the processor 608 of unit 400. The ratio is then compared to a predetermined table of values, for example comprising a plurality of empirical formulas, that convert the ratio to an SpO₂ value, i.e., the percentage saturation of hemoglobin with oxygen in the blood in the site that was tested. Such a table is typically based on calibration curves derived from healthy subjects at various SpO₂ levels. Typically a R/IR ratio of 0.5 equates to approximately 100% SpO₂, a ratio of 1.0 to approximately 82% SpO₂, while a ratio of 2.0 equates to 0% SpO₂.

In some embodiments, the wavelength of the light generated by light source 501 is sufficiently different from the transmitted red light of the blood oxygenation sensor module 700, such that the former only penetrates to the capillaries of the dermis skin layer, while the latter penetrates deeper into the subcutaneous tissue, enabling CRT and blood oxygenation measurements to be taken from the same vascular bed,

The blood oxygenation sensor module 700 may be based on other techniques for measuring blood oxygenation as known in the art, or on pulse oximetry techniques other than as described above, or on variations of the above pulse oximetry techniques, mutatis mutandis.

The modules 500 and 700 may be provided in the apparatus 100 very close to one another, such that the CRT data and the blood oxygenation data are provided for substantially the same anatomical part of the patient, in particular substantially the same vascular bed.

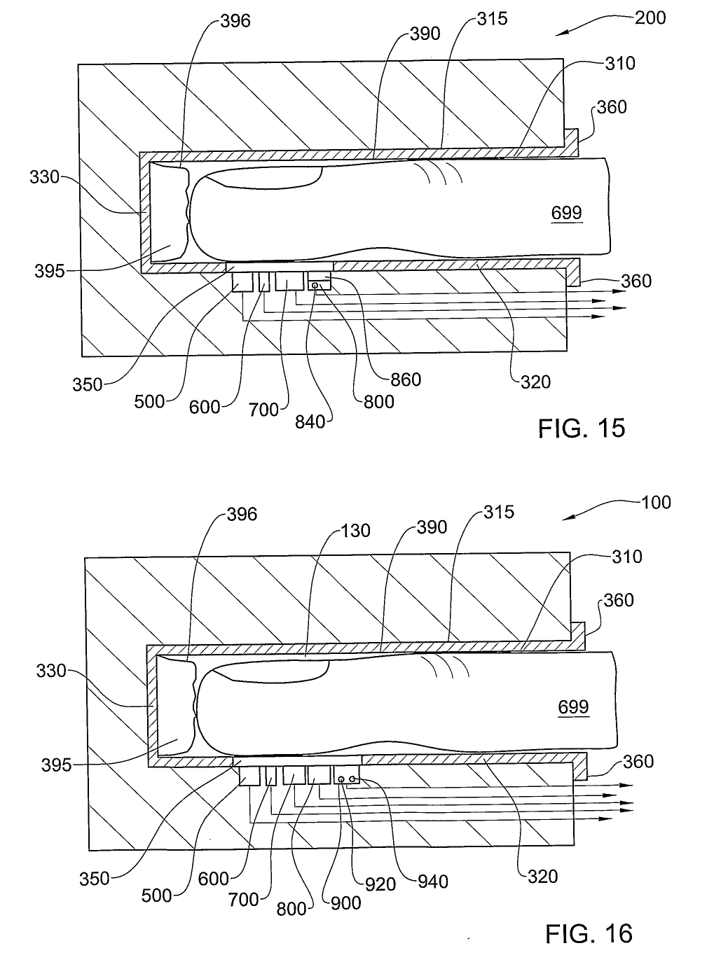

In a second embodiment of the sensing apparatus of invention, designated 200 in FIG. 15, the apparatus 200 comprises all the features and elements of apparatus 100 as described herein and variations thereof, mutatis mutandis, and thus includes CRT sensor module 500 and blood oxygenation sensor module 700, and optionally external temperature sensor 610 as described herein for the CRT sensor module 500 (or module 580) and blood oxygenation sensor module 700, external temperature sensor 610 respectively, of the first embodiment, mutatis mutandis. Thus, the system 10 may correspondingly comprise apparatus 200 rather than apparatus 100, mutatis mutandis.

In addition the apparatus 200 further comprises a blood pressure sensor module 800 for providing at least one cardio-respiratory parameter related to blood pressure, including for example blood pressure itself, pulse rate, systemic vascular resistance, or other cardio-respiratory parameters.

In one embodiment of the blood pressure sensor module 800, operation thereof is based on the Penaz method, for example in a manner similar to the operation of the commercially available Finometer and Portapres recorders. The blood pressure sensor module 800 comprises a plethysmograph 840 or any other means for measuring changes in volume, and thus arterial pulsation in the finger 699. The plethysmograph 840 is comprised in a pressure cuff 860 which is situated in the apparatus 200 such that the cuff 860 is pressing against an artery in the finger 699. The pressure applied by the cuff 860 is controllable, for example via processor 608, by means of the output of plethysmograph 840, which drives a servo-loop or the like to modify the cuff pressure such as to keep the output from the plethysmograph 840 substantially constant. Under these conditions, the artery is kept partially opened and the oscillations of pressure in the cuff 860 are monitored, for example by means of a strain gauge, transducer and so on, which feed their pressure output signals to processor 608. These oscillations often provide a measure of the intra-arterial pressure wave, and thus unit 400 can be suitably calibrated to provide an accurate estimate of changes in systolic and diastolic pressure from the pressure oscillations. Optionally, the changes in blood pressure may be stored and/or displayed by the unit 400.

Furthermore, the frequency of the pressure oscillations also provide a measure of the pulse rate of the patient, and thus unit 400 can be suitably calibrated to provide an accurate estimate of pulse rate from the pressure oscillations. Optionally, the pulse rate may be stored and/or displayed by the unit 400.

Furthermore, any suitable pulse contour analysis method may be applied, for example by means of processor 608, to analyse the waveform of the pressure oscillations of the pulse, which may provide a cardiac output such as a measure of the patient's systemic vascular resistance, which relates to the arterial stiffness or tone. Optionally, the data relating to systemic vascular resistance may be stored and/or displayed by the unit 400.

In a third embodiment of the sensing apparatus of invention, designated 300 in FIG. 16, the apparatus 300 comprises all the features and elements of apparatus 200 as described herein and variations thereof, mutatis mutandis, and thus includes CRT sensor module 500, blood oxygenation sensor module 700, and blood pressure sensor module 800, and optionally external temperature sensor 610 as described herein for the CRT sensor module 500 (or module 580) and blood oxygenation sensor module 700, blood pressure sensor module 800 and the external temperature sensor 610, respectively of the second embodiment, mutatis mutandis. Thus, the system 10 may correspondingly comprise apparatus 300 rather than apparatus 100 or apparatus 200, mutatis mutandis.

In addition the apparatus 300 further comprises a perfusion sensing module 900 (also referred to herein as a PU sensor module 900) for determining a perfusion based parameter or a perfusion dependent parameter, other than CRT, of the same anatomical part of the body as the other cardio-respiratory parameters are being monitored. Such a cardio-respiratory parameter is referred to herein as a PU parameter. In this embodiment, operation of the PU sensing module 900 is based on photoplethysmographic methods, and comprises a light emitter 920 having at least one LED, situated in the lumen 130 such that the light emitted from these emitters penetrates at least partly into the patient's finger 699. A photodetector 940 is located next to the emitter 920 overlaying the measuring site. The emitter 920 is adapted for emitting light in the visible or non-visible spectrum, and the photodetector 940 is adapted for receiving backscattered light from the target area on the patient's finger. The amount of light absorbed depends on the blood volume in the target area. The intensity of the reflected light, determined by the processor 608 provides an indication of the blood volume changes in the target area, and thus provides a measure of the blood perfusion. Processor 608 also controls operation of the PU sensing module 900, and means that may be incorporated in said processor 608 for this purpose are known.

When the temperature of a tested area of an organ or tissue decreases, the metabolism also decreases, and there is less blood flow taking part on the metabolism. Thus, a decrease in temperature results in a decrease in measured perfusion. Conversely, as temperature of the tested area increases, there may be an increase in the magnitude of the data obtained for the PU parameter. Suitable corrections to the perfusion measurements may be made to compensate for temperature effects. Such corrections may be based, for example, on empirical correlations that may be compiled accordingly. Alternatively, the user of the system, knowing the temperature of the patient as described above, can interpret the perfusion results accordingly.

Alternatively, the operating principle of PU sensing module 900 may be based on impedance phlebography methods and is similar to that described with respect to the module based on photoplethysmographic methods, mutatis mutandis, with the following differences. In this variation of the embodiment of the perfusion module 900, the emitter 920 and the photodetector 940 are replaced with a plurality of electrodes, mutatis mutandis, arranged in series such that the electrodes are in contact with the patient's finger at four different points along its length. The two outer electrodes are used to provide a suitable current, generated by processing and display unit 400, and this current may be rated, for example, at about 100 μA, at a frequency of between about 1 kHz and about 100 kHz. The two central electrodes, which define the measurement segment of the finger, detect a voltage. The changes in impedance between the two central electrodes is indicative of the volume changes in the finger, which in turn may be indicative of the changes in blood volume in the target area, and thus provides a measure of the blood perfusion. The processing and display unit 400 typically comprises a signal conditioner, form example a multi-channel DC amplifier, for scaling internal analog data originating from the central electrodes.

Alternatively, operation of the PU sensing module 900 is based on vascular ultrasonography methods, in particular Doppler ultrasonography methods, and is similar to that described with respect to the module based on photoplethysmographic methods, mutatis mutandis, with the following differences. In this variation of the embodiment of the perfusion module 900, the emitter 920 is replaced with a transducer adapted for generating ultrasonic waves, mutatis mutandis, typically with a frequency of about 2 MHz to about 10 MHz. The photodetector 940 is similarly replaced with a transducer for receiving the sound waves as they are reflected from the patient's finger, mutatis mutandis. Optionally, a single transducer may be used for transmitting and then receiving the reflected sound waves. The difference between the transmission frequency and the reflection frequency is determined by the processor 608 and represents a Doppler shift which in turn is indicative of the velocity of the blood in the target area, and thus blood perfusion there. Furthermore, the characteristics of the detected frequency shift indicate whether the blood flow is smooth and laminar or turbulent.

Alternatively, operation of the PU module 900 is based on Laser Doppler flowmetry (LDF) methods and is similar to that described with respect to the module based on photoplethysmographic methods, mutatis mutandis, with the following differences. In this variation of the embodiment of the perfusion module 900, the emitter 920 is replaced with a suitable optic fiber arrangement optically connected to a laser, and the photodetector 940 is replaced with another optical fiber for collecting backscattered light from the target area on the patient's finger. The reflected light is subjected to signal processing methods, by the processor 608, to determine the Doppler shift due to the moving red blood cells, and thereby provides a measure of the blood perfusion. Blood perfusion using LDF methods is proportional to the red blood cell perfusion or flux, and represents the transport of blood cells through microvasculature. The microvasculature perfusion, or red blood cell flux, may be defined as the product of the number of blood cells that are moving in the tissue sampling volume at the target area of the finger, and the mean velocity of these cells.

Alternatively, operation of the PU sensing module 900 may be based on other plethysmographic methods, including traditional volume change methods, or relatively newer methods such as using a Mercury strain gauge, in which the change in the electrical resistance of the gauge is indicative of the change in the volume of the finger, which in turn may be indicative of the change in blood volume, and thus of blood perfusion.

In a fourth embodiment of the sensing apparatus of invention, the sensing apparatus comprises all the features and elements of apparatus 100 as described herein and variations thereof for the first embodiment, mutatis mutandis, with the major difference that the blood oxygenation sensor module 700 is replaced with the blood pressure sensor module 800, as described for the second embodiment, mutatis mutandis, and thus includes CRT sensor module 500, and optionally external temperature sensor 610 as described herein for the CRT sensor module 500 (or module 580) and external temperature sensor 610 respectively, of the first embodiment, mutatis mutandis. Thus, the system 10 may correspondingly comprise this embodiment of the sensing apparatus rather than apparatus 100, mutatis mutandis.

In a fifth embodiment of the sensing apparatus of invention, the sensing apparatus comprises all the features and elements of apparatus 100 as described herein and variations thereof for the first embodiment, mutatis mutandis, with the major difference that the blood oxygenation sensor module 700 is replaced with the PU sensor module 900, as described for the third embodiment, mutatis mutandis, and thus includes CRT sensor module 500, and optionally external temperature sensor 610 as described herein for the CRT sensor module 500 (or module 580) and external temperature sensor 610 respectively, of the first embodiment, mutatis mutandis. Thus, the system 10 may correspondingly comprise this embodiment of the sensing apparatus rather than apparatus 100, mutatis mutandis.

In a sixth embodiment of the sensing apparatus of invention, the sensing apparatus comprises all the features and elements of apparatus 100 as described herein and variations thereof for the first embodiment, mutatis mutandis, with the major difference that the CRT sensor module 500 is replaced with the blood pressure sensor module 800, as described for the second embodiment, mutatis mutandis, and thus includes blood oxygenation sensor module 700, and optionally external temperature sensor 610 as described herein for the blood oxygenation sensor module 700 and external temperature sensor 610 respectively, of the first embodiment, mutatis mutandis. Thus, the system 10 may correspondingly comprise this embodiment of the sensing apparatus rather than apparatus 100, mutatis mutandis.

In a seventh embodiment of the sensing apparatus of invention, the sensing apparatus comprises all the features and elements of apparatus 100 as described herein and variations thereof for the first embodiment, mutatis mutandis, with the major difference that the CRT sensor module 500 is replaced with the PU sensor module 900, as described for the third embodiment, mutatis mutandis, and thus includes blood oxygenation sensor module 700, and optionally external temperature sensor 610 as described herein for the blood oxygenation sensor module 700 and external temperature sensor 610 respectively, of the first embodiment, mutatis mutandis. Thus, the system 10 may correspondingly comprise this embodiment of the sensing apparatus rather than apparatus 100, mutatis mutandis.

In an eighth embodiment of the sensing apparatus of invention, the sensing apparatus comprises all the features and elements of apparatus 100 as described herein and variations thereof for the first embodiment, mutatis mutandis, with the major difference that the CRT sensor module 500 is replaced with the PU sensor module 900, as described for the third embodiment, mutatis mutandis, and blood oxygenation sensor module 700 is replaced with the blood pressure sensor module 800, as described for the second embodiment, mutatis mutandis, and thus optionally includes external temperature sensor 610 as described herein for external temperature sensor 610, of the first embodiment, mutatis mutandis. Thus, the system 10 may correspondingly comprise this embodiment of the sensing apparatus rather than apparatus 100, mutatis mutandis.

In a ninth embodiment of the sensing apparatus of invention, the sensing apparatus comprises all the features and elements of apparatus 200 as described herein and variations thereof for the second embodiment, mutatis mutandis, with the major difference that the blood pressure sensor module 800 is replaced with the PU sensor module 900, as described for the third embodiment, mutatis mutandis, and thus also includes CRT sensor module 500, blood oxygenation sensor module 700, and optionally external temperature sensor 610 as described herein for the CRT sensor module 500 (or module 580) blood oxygenation sensor module 700, and external temperature sensor 610 respectively, of the second embodiment, mutatis mutandis. Thus, the system 10 may correspondingly comprise this embodiment of the sensing apparatus rather than apparatus 100, mutatis mutandis.

In a tenth embodiment of the sensing apparatus of invention, the sensing apparatus comprises all the features and elements of apparatus 200 as described herein and variations thereof for the second embodiment, mutatis mutandis, with the major difference that the blood oxygenation sensor module 700 is replaced with the PU sensor module 900, as described for the third embodiment, mutatis mutandis, and thus also includes CRT sensor module 500, blood pressure sensor module 800, and optionally external temperature sensor 610 as described herein for the CRT sensor module 500 (or module 580), blood pressure sensor module 800, and external temperature sensor 610 respectively, of the second embodiment, mutatis mutandis. Thus, the system 10 may correspondingly comprise this embodiment of the sensing apparatus rather than apparatus 100, mutatis mutandis.

In an eleventh embodiment of the sensing apparatus of invention, the sensing apparatus comprises all the features and elements of apparatus 200 as described herein and variations thereof for the second embodiment, mutatis mutandis, with the major difference that the CRT sensor module 500 is replaced with the PU sensor module 900, as described for the third embodiment, mutatis mutandis, and thus also includes blood oxygenation sensor module 700, blood pressure sensor module 800, and optionally external temperature sensor 610 as described herein for the blood oxygenation sensor module 700, blood pressure sensor module 800, and external temperature sensor 610 respectively, of the second embodiment, mutatis mutandis. Thus, the system 10 may correspondingly comprise this embodiment of the sensing apparatus rather than apparatus 100, mutatis mutandis.

Thus, according to one aspect of the invention, a sensing apparatus, and corresponding system, may be provided for measuring nay combination of two, three four or more different cardio-respiratory parameters, and optionally temperature, of an anatomical part of a patient.

For all the embodiments of the system 10, the processing and display unit 400 may further comprise a display 609 for displaying the results. The display 609 may comprise a screen, and may incorporate “touch screen” technology, that allows commands to be conveyed therefrom to the processor 608 by touching the screen where certain icons, menus, etc., may appear. Alternatively, or additionally, display 609 may comprise a printer.

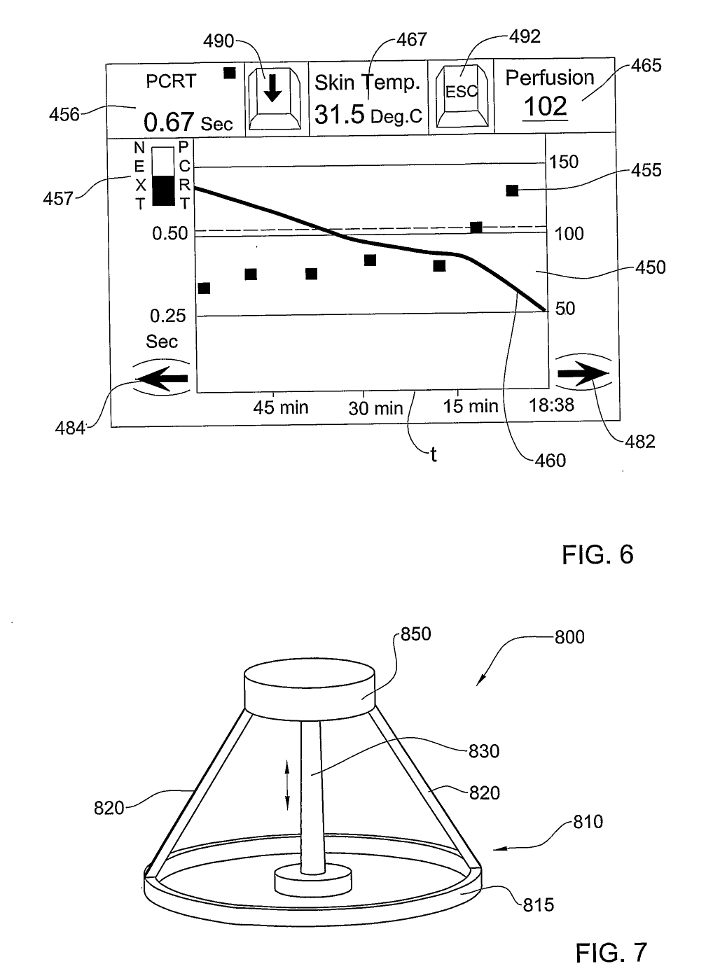

FIG. 6 illustrates one possible format for displaying test results relating to the CRT and PU parameters simultaneously, on the display 609 in real time, for example as may be obtained with the aforementioned fifth embodiment of the sensing apparatus. A graph 450 is provided at the center of the screen, in which the x-axis represents elapsed time t from the start of the test, which in the illustrated example was at 18:38. As time progresses, CRT measurements are conducted at preset intervals, in the example every 7-9 minutes, and are displayed as points 455 on the screen. The left hand y-axis displays the CRT scale. The current value 456 of CRT is also displayed in alphanumeric characters above the graph. An icon 457 also shows when the next CRT test will commence as a bar chart which “fills up” as the time for the next test approaches. Perfusion measurements are conducted continuously, or at shorter intervals, in the order of a few seconds, for example, and are displayed as a continuous or semi continuous curve 460 overlaid over the CRT results. The right hand y-axis displays the perfusion units scale The current value 465 of the perfusion units is also displayed in alphanumeric characters above the graph, together with the measured current value of skin temperature 467. When the time period for which the test is being conducted exceeds the reading on the scale, in the illustrated example after about 60 minutes, the graph scrolls continuously or semi continuously to the right and the current status of perfusion and CRT is located at the left end of the graph. Thus, the last 60 minutes of the test are readily shown, no matter how long the test has been going on for. If the user wishes to inspect test results prior to the current time window on the screen, this may be done by means of scrolling icons 482 and 484.

Icon 490 enables the user to manually initiate the CRT test at any time during the monitoring process, i.e., even while the system 10 is in an automatic mode of operation. Icon 492 enable the user to exit from the monitoring screen to a user menu, in which the user may choose various operations such as for example, restart a test, print results, and so on.

In a similar manner, mutatis mutandis, any combination of cardio-respiratory parameters can be suitably displayed, for example in real time, according to number and the specific type of parameters being monitored by the sensing apparatus and system. For example, the display 609 may display CRT data and/or blood oxygenation (e.g. SpO2) data, and/or PU data, and/or blood pressure data (e.g., pulse rate and/or blood pressure and/or systemic vascular resistance, etc.), in an appropriate manner, for example in a manner that facilitates diagnosis of the cardio-respiratory state of a patient, for example whether the patient is in early shock, suffering from dehydration which may lead to shock, or any other distortion of the general cardio-respiratory state of the patient.

Referring to FIGS. 2, 14 and to FIGS. 15 and 16, the sensing apparatus 100, 200 and 300, respectively, according to these embodiments or any other embodiments of the invention, optionally further comprise a sheath 315 that is worn over the finger 699 when the finger is inserted into the lumen 130. The sheath 315 is preferably disposable, and thus made from an economically inexpensive material, wherein the cost of such a sheath is substantially well below the cost of other components of the sensing apparatus 100. Alternatively, the sheath 315 may be reusable, and thus made from a suitable material that may be cleaned, and preferably sterilized between patients. The sheath 315 is constructed as an elongate integral item, wherein an upper part 310 folds over a lower part 320 by means of a deformable end portion 330 therebetween, in overlying relationship, defining an inner space 340 for directly accommodating the finger 699, and releasably locked in this relationship via suitable locking arrangement (not shown) when worn over the finger.

The sheath 315 comprises an aperture 350 which is situated on the sheath such as to allow access to the CRT sensor module 500 and/or the blood pressure sensor module 800 and/or some embodiments of the PU sensor module 900, to contact the skin surface of the finger 699 when the apparatus 100 is in operation, and to operate as described herein. Optionally, the same aperture 350 serves to allow optical communication between optical components of other modules, such as some embodiments of the PU sensor module 900 and the blood oxygenation sensor module 700, and the finger, for example. To aid in this alignment, the sheath 315 comprises a flange 360 that abuts against the outside of the apparatus 100 when the sheath 315 is fully inserted therein, and may further comprise a key (not shown) to ensure that the sheath is always inserted in the correct orientation with respect to the lumen 130. Thus, the sheath 315 may be locked over a finger such that the aperture 350 and windows 360 are on the desired locations on the finger 699, thereby ensuring that these areas will be subjected to the CRT and perfusion measurements when the sheathed finger is inserted into the apparatus 100.

Alternatively, the sheath 315 may further comprise, in addition to aperture 350, one or more optically transparent windows which may be located such as to be in registry with the optical components of some modules, such as some embodiments of the PU sensor module 900 and/or the blood oxygenation sensor module 700, when the sheath 315 is fully received in the lumen 130.

The sheath 315 also enables patients with widely varying finger sizes to use the same apparatus 100. For example, for infants and babies, a sheath having a substantially thicker wall 390 may be used, and having a plug 395 at the end portion 330 to ensure a snug fit between the finger and the sheath, and between the sheath and the lumen 130. The plug 395 preferably comprises a recess 396 for accommodating the potentially projecting portion of the nail of finger 699, which thus avoids time being wasted in trimming nails when such occasions arise. Thus, a number of different sized sheaths may be provided for use with the same sensing apparatus, each sheath having the same external dimensions when locked over a finger, but different internal dimensions according to the age, sex and size of the patient.

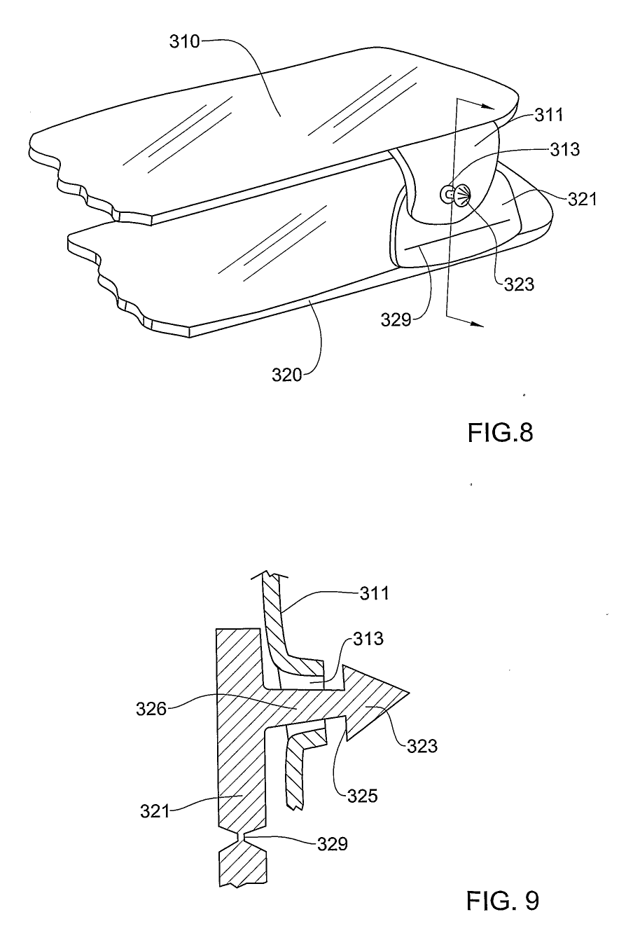

Optionally, and when the sheath is disposable, means may be provided for irreparably damaging the sheath after it has been used by one patient, to prevent it from being used by another patient. Such means may be comprised, for example, in the aforesaid locking means, which rather than being reversibly lockable may be locked, but not unlocked, and to remove the sheath the lock has to be destroyed, preventing the sheath from being used again. Referring to FIGS. 8 and 9, the locking means comprises an upper flap 311 comprised on either side of said upper part 310, and comprising an aperture 313. The locking means also comprises a lower flap 321 comprised on either side of said lower part 320, and comprising a stud 323 that is designed to penetrate through aperture 313 when the locking means are closed. The leading edge of the stud is rounded or pointed, and thus allows penetration through the aperture 313, which is resilient, deformable and/or otherwise configured to allow passage therethrough, even though the width of the stud is larger than that of the aperture. However, the latter fact, coupled with the flat nature of the base 325 of the stud 323 prevents the stud from being removed again via the aperture, unless a high enough force is applied. The neck 326 of the stud 323 can be designed to shear off when such is force is applied, thereby destroying the locking means. Alternatively, such means could comprise, for example, a weakened tear line 329 along one of the flaps, say the lower flap 321, which tears off when a relatively small predetermined separating force is applied between the upper part 310 and the lower part 320. The end 330 is designed to spring back the upper part 310 and the lower part 320 in the absence of the locking means being in engagement.

Sensing system 10 may further include receiving and transmitting circuits to enable wireless exchange of data and control commands required for cardio-respiratory measurements, including for example, CRT, and/or blood oxygenation, and/or blood pressure and/or PU measurements. Wireless connection makes feasible a single processing and display unit 400 to control and monitor several sensing apparatuses 100 (and/or apparatuses 200 and/or apparatuses 300 according to any embodiments thereof), each being attached to a different patient. Each sensing apparatus may be identified by a unique code assigned to it, to eliminate false associations between processed data and a patient. Furthermore, such wireless communication also enables the measurements from each sensing apparatus to be sent, via the internet, for example, or any other data communication network, to a processing unit that is remote from the sensing apparatus. In other words, the sensing functions of the sensing apparatus may be done on site, wherever the patient is located, whereas the processing and display functions of the unit 400 may be carried out at a different location. Thus, while ambulance or paramedic staff may attach the sensing apparatus to a patient at the scene of an accident, for example, a doctor many miles away, either at the hospital or on the way to the accident scene, for example, can view the cardio respiratory results via an internet or other wireless connection, and may thus be able to advise the paramedics on the emergency procedure to administer.

A cardio-respiratory diagnostic system in accordance with the invention is a non-invasive diagnostic tool which determines the cardio-respiratory state of the patient, including for example the degree to which a patient may be in a state of shock, making it possible for a clinician to prescribe a treatment that may save the patient's life. This instrument affords the field of medicine with a plurality of vital signs, including one or more of pulse rate, body temperature and often blood pressure and CRT, and other signs such as respiratory rate may be complementary.

In the method claims that follow, alphanumeric characters and Roman numerals used to designate claim steps are provided for convenience only and do not imply any particular order of performing the steps.

Finally, it should be noted that the word “comprising” as used throughout the appended claims is to be interpreted to mean “including but not limited to”.

While there has been shown and disclosed example embodiments in accordance with the invention, it will be appreciated that many changes may be made therein without departing from the spirit of the invention.

1-76. (canceled)

77. Apparatus for providing data indicative of cardio-respiratory state of a patient, the apparatus comprising at least two cardio-respiratory sensor modules for providing at least two cardio-respiratory parameters, including:—
first sensor module for measuring a first cardio-respiratory parameter of said patient, wherein said first cardio-respiratory parameter is capillary refill time (CRT);
second sensor module for measuring a second cardio-respiratory parameter of said patient, different from said first cardio-respiratory parameter, wherein said second cardio-respiratory parameter is any one of blood oxygenation state, a peripheral perfusion parameter (PU) other than capillary refill time, blood pressure, pulse rate, systemic vascular resistance;
wherein said apparatus is adapted for measuring said first cardio-respiratory parameter and said second cardio-respiratory parameter at a same anatomical part of said patient.

78. Apparatus according to claim 77, wherein said apparatus further comprises a third cardio-respiratory sensor module for measuring at said same anatomical part at least one third cardio-respiratory parameter of said patient different from said first or second cardio-respiratory parameters.

79. Apparatus according to claim 78, wherein said apparatus further comprises a fourth cardio-respiratory sensor module for measuring at said same anatomical part at least one fourth cardio-respiratory parameter of said patient different from said first, second or third cardio-respiratory parameters.

80. Apparatus according to claim 78, wherein each said cardio-respiratory sensor is configured for monitoring a different one of any of the following cardio-respiratory parameters: capillary refill time (CRT); a peripheral perfusion parameter other than CRT; blood oxygenation level; blood pressure; pulse rate; systemic vascular resistance.

81. Apparatus according to claim 79, wherein each said cardio-respiratory sensor is configured for monitoring a different one of any of the following cardio-respiratory parameters: capillary refill time (CRT); a peripheral perfusion parameter other than CRT; blood oxygenation level; blood pressure; pulse rate; systemic vascular resistance.

82. Apparatus according to claim 77, wherein at least two said cardio-respiratory sensors are configured for measuring corresponding cardio-respiratory parameters with respect to a common vascular bed on said same anatomical part.

83. Apparatus according to claim 77, wherein said first cardio-respiratory sensors comprises a CRT sensor module configured for monitoring a capillary refill time (CRT), said CRT sensor module comprising:
i) means for illuminating a skin area comprised in said same anatomical part to be gauged for wavelength with a light from a light source;
ii) means for filtering out background noises and light to obtain a base-line measurement; and
iii) means for comparing the wavelength of light received from the skin area with the base-line measurement, thereby determining the filling time of blood vessels in said area.

84. Apparatus according to claim 77, wherein said first cardio-respiratory sensors comprises a CRT sensor module configured for monitoring a capillary refill time (CRT), said CRT sensor comprising:
i) a light source for illuminating a skin area of the patient's skin overlying blood vessels with light at a first wavelength, said skin area having an original color, a light sensor for intercepting light at a second wavelength obtained from said skin area or at a depth within said skin area and generating a first signal having a magnitude which corresponds to the second wavelength, said second wavelength representing a level of reflection from blood vessels subjacent said skin area;
ii) a filter for filtering said first electrical signal and for rejecting unwanted electrical signals originating in interfering light, and for producing a second signal, whose amplitude is proportional to the amplitude of said filtered first signal;
iii) means for storing the amplitude value of said second signal which corresponds to said original color;
iv) a transducer for applying pressure on said skin area, and for obtaining an amplitude of the second signal which corresponds to maximum whitening of said skin area.

85. Apparatus according to claim 84, further comprising a processor for processing data collected by said transducer and for measuring the filling time of blood vessels after releasing said pressure.

86. Apparatus according to claim 85, wherein said measuring the filling time of blood vessels after releasing said pressure is provided by analysing a rate of change of light intensity of said second wavelength with respect to elapsed time after releasing said pressure.

87. Apparatus according to claim 84, further comprising a suitable mechanism for automatically applying and releasing said pressure.

88. Apparatus according to claim 84, further comprising a first temperature sensor for sensing skin temperature of a second skin area close to said first mentioned skin area, wherein said second skin area is substantially unaffected by heat effects generated by said apparatus.

89. Apparatus according to claim 88, further comprising a second temperature sensor for sensing skin temperature of said first mentioned area, wherein said first mentioned skin area is substantially unaffected by heat effects generated by said apparatus.

90. Apparatus according to claim 77, wherein said second cardio-respiratory sensors is a blood oxygenation (BO) sensor module configured for monitoring blood oxygenation state, wherein operation of said BO sensor module is based on pulse oximetry techniques.

91. Apparatus according to claim 90, wherein said BO sensor module is adapted for measuring SpO2 and comprises at least one emitter for emitting red and infra red light, and at least one photodetector for receiving backscattered light from a target area of said patient at said anatomical part.

92. Apparatus according to claim 91, wherein said at least one photodetector is adapted for operating according to any one of a transmission method or a reflectance method, wherein in said transmission method, said at least one emitter and said at least one photodetector are in opposed relationship with respect to an extremity during operation of said apparatus, and wherein in said reflectance method, said at least one photodetector is adapted for operating according to a reflectance method, and wherein said at least one emitter and said at least one photodetector are in adjacent relationship.

93. Apparatus according to claim 77, wherein said second cardio-respiratory sensors is a peripheral perfusion (PU) sensor module configured for monitoring a peripheral perfusion parameter other than CRT.

94. Apparatus according to claim 93, wherein operation of said PU sensor module is based on any one of the following:—
photoplethysmographic techniques, and wherein said PU sensor module comprises at least one emitter for emitting light in the visible or non visible spectrum, and at least one photodetector for receiving backscattered light from a target area of said patient;
vascular ultrasonography techniques, and wherein said PU sensor module comprises at least one transducer for generating suitable ultrasonic waves, and at least one transducer for receiving sound waves reflected from a target area of said patient;
Doppler flowmetry techniques, and wherein said PU sensor module comprises at least one optic fiber operatively connected to a laser for emitting light, and at least one optical fiber for receiving backscattered light from a target area of said patient;
suitable plethysmographic techniques.

95. Apparatus according to claim 77, wherein said second cardio-respiratory sensors is a blood pressure (BP) sensor module configured for monitoring at least one of blood pressure, pulse rate, systemic vascular resistance.

96. Apparatus according to claim 95, wherein operation of said BP sensor module is based on suitable Penaz techniques.

97. Apparatus according to claim 95, wherein said BP sensor module comprises a plethysmograph and a pressure cuff, wherein a pressure applied by the cuff is controllable using an output of plethysmograph such as to maintain the output from the plethysmograph substantially constant.

98. Apparatus according to claim 77, wherein said apparatus further comprises a body temperature sensor for measuring a body temperature of said patient at said same anatomical part.

99. Apparatus according to claim 78, wherein said second cardio-respiratory sensor module comprises a blood oxygenation (BO) sensor module configured for monitoring blood oxygenation state; and wherein said third cardio-respiratory sensor module comprises a blood pressure (BP) sensor module configured for monitoring at least one of blood pressure, pulse rate, systemic vascular resistance

100. Apparatus according to claim 77, wherein said apparatus is adapted for accommodating a finger of said patient, said finger comprising said same anatomical part.

101. A system for providing data indicative of cardio-respiratory state of a patient comprising:—
apparatus as defined in claim 77; and
user interface for enabling data relating to at least two said cardio-vascular parameters obtained from said apparatus to be at least one of processed and displayed.

102. A system according to claim 101, wherein said apparatus is operatively connected to said user interface via at least one of a suitable cable and a suitable wireless connection.

103. A system according to claim 102, wherein said wireless connection is via the Internet.

104. A system according to claim 101, wherein said apparatus is integrated with said user interface in the form of a handheld device.

105. A method for providing data indicative of cardio-respiratory state of a patient comprising measuring at least two cardio-respiratory parameters of said patient, wherein one said cardio-respiratory parameter is capillary refill time (CRT) and wherein another said cardio-respiratory parameter is any one of blood oxygenation state, a perfusion parameter (PU) other than capillary refill time (CRT), blood pressure, pulse rate, systemic vascular resistance, herein said at least two cardio-respiratory parameters are different one from the other and are measured at a same anatomical part of said patient.

106. A sheath for use with a sensing device, wherein the sheath is adapted to be worn over a finger, said sheath comprising at least one window for allowing communication between an inside and an outside of the sheath, wherein the sheath is adapted for becoming unusable as a sheath after being removed from a finger.

107. A sheath according to claim 106, wherein said sheath is made from a disposable material.

108. A sheath according to claim 106, wherein said sheath comprises an upper portion foldable over a lower portion in overlying relationship by means of a deformable first end portion therebetween, such as to define an opening at a second end thereof opposed to said first end, and an inner space for accommodating a finger.
